(19) United States
(12) Patent Application Publication
(10) Pub. No.: US 2026/0031232 A1
(43) Pub. Date: Jan. 29, 2026

(54) ARTIFICIAL INTELLIGENCE BASED METHOD AND APPARATUS FOR AUTOMATED PREPARATION, ANALYSIS, AND SCREENING FOR CANCER SUSPICION

(71) Applicant: Alfredo R. Zarate, Bethesda, MD (US)

(72) Inventor: Alfredo R. Zarate, Bethesda, MD (US)

(21) Appl. No.: 18/781,672

(22) Filed: Jul. 23, 2024

Related U.S. Application Data
(63) Continuation-in-part of application No. 16/945,278, filed on Jul. 31, 2020, now abandoned.

(51) Int. Cl. G16H 50/30; G01N 1/30; G01N 35/00; G01N 35/04; G01N 35/10; G16H 10/60; G16H 15/00; G16H 30/40

(52) U.S. Cl. CPC G16H 50/30; G01N 1/30; G01N 35/00029; G01N 35/00732; G01N 35/0099; G01N 35/04; G01N 35/10; G16H 10/60; G16H 15/00; G16H 30/40; G01N 2035/00138; G01N 2035/00495; G01N 2035/0429; G01N 2035/1039

(57) ABSTRACT

The present invention relates to automation of urine analysis or urinalysis (UA) with conventional cytopathological assessment tests and methods using an artificial intelligence (AI) based urine diagnostic apparatus or device or system and methods employing said apparatus or device or system for fully automated preparation of raw samples by automating and streamlining conventional laboratory processes and examination of urine samples, automated data collection, automated analysis of data using digital image recognition, and finally, automated screening for cancer suspicion in a non-invasive, non-human or pre-pathologist intervention-based automatic examination and analysis of such samples using AI based on said data analysis for preparing reports intimating to the subject or patient automated negation if no abnormal cells are detected/identified, or to forward suspicious samples from the subject or patient to confirm the possibility of suspicion for cancer risk of cancers routinely and otherwise detected based in examination of urine samples by a trained pathologist.

### FIELD OF THE INVENTION

The present invention relates generally to the field of urinalysis, cytopathological assessment methods using an artificial intelligence (AI) based urine diagnostic apparatus or device and methods employing said apparatus or device. More specifically, the present invention relates to examination of urine samples using an artificial intelligence (AI) urine diagnostic apparatus or device and methods employing said apparatus or device for automated preparation of raw samples based in conventional laboratory processes, automated data collection, automated analysis of data, and automated screening for cancer suspicion based on said data for automated negation or suspicion for cancer risk of cancers commonly detectable based in urine samples.

### PRIORITY

This application is a continuation-in-part application of U.S. application Ser. No. 16/945,278 filed on Jul. 31, 2020 the contents of which are incorporated herein by reference in their entirety.

### INCORPORATION BY REFERENCE

All publications, patents, and patent applications mentioned in this specification are herein incorporated by reference in their entirety to the same extent as if each individual publication or patent application was specifically and individually indicated to be incorporated by reference.

### BACKGROUND OF THE INVENTION

Kidney cancer is among the ten most common cancers in both men and women and its occurrence has been rising since the 1990s (1). It was estimated that in 2019 about seventy-four thousand kidney and renal pelvis cancers will occur and that about fifteen thousand people will die from this disease. Men have a lifetime risk for developing kidney cancer of 1 in 48 and women of 1 in 83. Likewise, bladder cancer is the ninth most frequently diagnosed cancer worldwide, more than thousand cases are diagnosed yearly, and ranks thirteenth in terms of death (2).

Urine cytology is part of the standard triad to diagnose renal and urothelial carcinoma, along with cystoscopy and imaging studies. However, these studies are usually performed whenever there is clinical evidence of disease, usually hematuria lower urinary tract symptoms, a history of occupational exposure, or the follow-up of patients with previous diagnosis of urothelial cancer. In addition, the cytological examination of urine samples unstained (3-4) or stained (5-14) and measuring soluble or cell attached cancer biomarkers in urine samples (15-22), all useful tools for the diagnosis of carcinoma of the kidney and the urinary tract, are time consuming and labor intensive methods, the procedure to prepare the cytology samples may vary among different labs, i.e. volume of sample, duration, rpms and method of centrifugation, the samples must be examined by cytopathologists (or by a cytogeneticist in the FISH method) that may have different degrees of expertise, and all of them have a high cost and cannot be used to screen a large number of samples.

The Paris system (10) standardizes the urine cytology reporting and increases the sensitivity of diagnosis of high-grade urothelial carcinoma (HGUC) (9, 11) by reducing the rate of indeterminate atypical diagnoses (8) but it may increase the cases in the atypical category (12), and there is inter-observer variability of findings (13). Other methods, i.e. the Fluorescent In Situ Hybridization (FISH) method has been found to have higher sensitivity than standard cytology with pap method for low grade urothelial carcinoma (UC) (18) or similar sensitivity (15-17). Newer tests have been developed, i.e. using CellDetect staining (5), Hemocolor staining (24), and measuring other urine biomarkers, i.e. ImmunoCyt (CEA), Nuclear matrix protein 22 (NMP22), Uro Vysion (chromosomal changes) (15-17) but they are also cumbersome and expensive. Besides, there is variability in the preparation of the cytology sample (i.e. volume of urine), the methods of processing the sample, (i.e. ThinkPrep, SurePath), and centrifugation, i.e. Cytospin (25-26).

Even though urine cytology has a low sensitivity of 38% and a high specificity of 98% to diagnose high growth urothelial carcinoma and a lower sensitivity to diagnose low grade tumors (5, 24, 7), the likelihood of these diagnoses will improve if the tests is performed often, i.e. every 4-6 months, at the same time a urinalysis, or if an excess of cells are found in a routine urinalysis, and if the sensitivity of the test could be improved by centrifuging a larger sample of urine, i.e. 50 mL-100 mL. The UA is one of the most frequently used tests, in the US in 1981 it was done in 50M occasions among 150M outpatient visits (29) and in 2016 it was likely done in a higher number, maybe 600M times, among 990M of outpatient visits (30). However, the current routine urinalysis (UA) is limited in terms of diagnosis for cancer risk suspicion and often times, even the negation of said cancer risk susceptibility in samples submitted for UA is heavily fraught with false positives and false negatives. For instance, the UA falls short in terms of taking note of abundant transitional epithelial cells which are rarely seen in a urinalysis sediment and its presence requires to rule/out neoplasia or urinary tract infections (31-33) or if red blood cells (rbcs) are identified in the urinalysis sediment, either dysmorphic or isomorphic, a cytological examination to identify malignance cells needs to be simultaneously and additionally done. Thus, the UA as used conventionally is not very efficient on its own, has a lot of variation and human error associated with it is quite variable and may be high depending on the varying expertise of the examining pathologist, it is time consuming, it is not as accurate as it can be with the use of automation by taking out human error, it may lead to unnecessary downstream medical expenses for a false positive and yet miss out on potential cancer cases owing to false negative results driven by variation in capability and training of pathologists, for instance. There is, hence, a problem of standardization, reproducibility, reliability with the current UA, highlighting a need to complement the reach and capacity of well-trained and experienced pathologists who have limited time and resources to properly examine and assess smaller number of samples and benefit a small number of potential patients only. Also, the variation in which a pathologist (expert or novice, for instance) examines and assesses progression of cancer can further lead to problems, raising a need for consistency and reliability.

The present invention addresses the above-discussed problem and need for reducing the requirement of human intervention which as discussed above is fraught with human error, false positives and false negatives in the initial stages of diagnosis of suspicion of cancer risk or negation thereof itself, which the apparatus, system, and method as disclosed in the present invention by employing the help of automation and artificial intelligence (AI) overcomes. The present invention advantageously incorporates said automation and AI into the UA to reduce human intervention and error to decrease the number of false positives and false negatives, increasing efficiency, reliability, providing a standardized method and means in the initial stages of deducing a probability of cancer risk suspicion or negating any such suspicion without human intervention to make the overall process more proficient, timesaving, laborsaving, economical, and competent. It would complement and streamline downstream additional processes of identification of cancer and its type by a trained expert pathologist of cancers which are routinely and conventionally diagnosed by the route of UA.

### SUMMARY OF THE INVENTION

The following listing of embodiments is a nonlimiting statement of various aspects of the invention. Other aspects and variations will be evident in light of the entire disclosure.

The present invention relates to urine analysis or urinalysis (UA) with conventional cytopathological assessment tests and methods using an artificial intelligence (AI) based urine diagnostic apparatus or device or system and methods employing said apparatus or device or system for fully automated preparation of raw samples by automating and streamlining conventional laboratory processes and examination of urine samples, automated data collection, automated analysis of data using digital image recognition, and finally, automated screening for cancer suspicion in a non-invasive, non-human or pre-pathologist intervention-based automatic examination and analysis of such samples using AI based on said data analysis for preparing reports intimating to the subject or patient automated negation if no abnormal cells are detected/identified, or to forward suspicious samples from the subject or patient to confirm the possibility of suspicion for cancer risk of cancers routinely and otherwise detected based in examination of urine samples by a trained pathologist. The present invention thus provides means and methods that automate report generation for negation of suspicion of cancer risk based on UA at the outset, without human intervention based in AI training and automation, or possibility of cancer risk which forms the basis for further examination, assessment, and analysis with human intervention such as by a trained pathologist to confirm the suspicion (and negation of cancer risk, if needed). This is based on the Paris System for identification of various cancer cells and types that are generally identified clinically via urinalysis (UA) of urine samples by such pathologists for cancers that include, kidney and renal pelvis cancers, bladder cancer, renal and urothelial carcinoma, carcinoma of the kidney and the urinary tract, high-grade urothelial carcinoma, etc. By employing and incorporating AI the present invention makes the overall process more efficient, faster, concentrating only on further downstream processing of positively suspected cases thereby reducing the load on pathologists while the automation negates and removes negative samples in which no abnormal cells are detected/identified that would have been otherwise identified as false positives. It is due to the load of samples prior to the instant automation approach along with human error of pathologists that used to lead to false negatives for samples identified in the present invention as having suspicion of cancer risk thereby missing such patients that may lead to drastic and life-threatening effects for such patients. Thus, the present invention is highly advantageous and efficient for detection of suspicion of cancer risk and negation thereof without much human intervention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The accompanying drawings are included to provide a further understanding of the invention, and are incorporated in and constitute a part of the present invention and, together with the description, serve to explain the principle of the invention.

In the drawings,

FIG. 1A depicts an embodiment of present invention and illustrates an exemplary schematic view of the apparatus and system for use in the method of screening for cancer risk suspicion in a subject undergoing urine analysis or urinalysis.

FIG. 1B depicts an embodiment of present invention and illustrates a more focused exemplary schematic view of the apparatus and system for use in the method of screening for cancer risk suspicion in a subject undergoing urine analysis or urinalysis.

FIG. 2 illustrates an embodiment of present invention and illustrates an exemplary image of an analyte prepared via the overall method as disclosed in the present invention, enhanced via microscope.

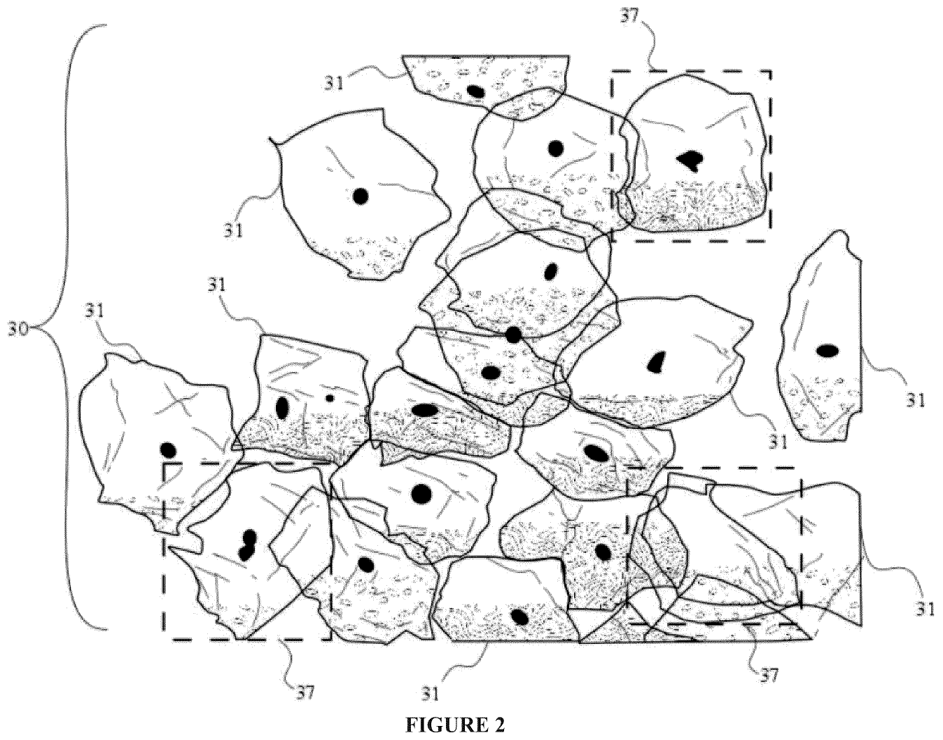

FIG. 3 illustrates an embodiment of present invention and illustrates an exemplary schematic providing a flowchart illustrating one of the embodiments for an overall method of the present invention.

FIG. 4 illustrates an embodiment of present invention and illustrates an exemplary schematic providing a flowchart illustrating a sub-process for assembling and collating multiple results of multiple consecutive iterations of the overall method in one of the embodiments of the present invention.

FIG. 5 illustrates an embodiment of present invention and illustrates an exemplary schematic providing a flowchart illustrating another one of the embodiments for an overall method of the present invention.

### DETAILED DESCRIPTION OF THE INVENTION

Detailed embodiments of the present invention are disclosed herein. However, it is to be understood that the disclosed embodiments are merely exemplary of the present invention, which may be embodied in various systems. Therefore, specific details disclosed herein are not to be interpreted as limiting, but rather as basis for teaching one skilled in the art to variously practice the present invention.

All illustrations of the drawings are for the purpose of describing selected versions of the present invention and are not intended to limit the scope of the present invention.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

Although any methods and materials similar or equivalent to those described herein can be used in the practice of the present invention, the exemplary methods, devices, and materials are described herein. For the purposes of the present disclosure, the following terms are defined below. Additional definitions are set forth throughout this disclosure.

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a microbe, a microbial formulation, a pharmaceutical composition, and/or a method that “comprises” a list of elements (e.g., components, features, or steps) is not necessarily limited to only those elements (or components or steps), but may include other elements (or components or steps) not expressly listed or inherent to the microbe, microbial formulation, pharmaceutical composition and/or method. Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As used herein, the term “and/or” when used in a list of two or more items, means that any one of the listed items can be employed by itself or in combination with any one or more of the listed items. For example, the expression “A and/or B” is intended to mean either or both of A and B, i.e., A alone, B alone or A and B in combination. The expression “A, B and/or C” is intended to mean A alone, B alone, C alone, A and B in combination, A and C in combination, B and C in combination or A, B, and C in combination.

As used herein, the terms “subject,” “patient” and “individual” are used interchangeably herein to refer to a vertebrate, including mammals and humans. A “subject,” “patient” or “individual” as used herein, includes any animal that exhibits pain that can be treated with the compositions or formulations or systems, and methods contemplated herein, and that includes laboratory animals, farm animals, and domestic animals or pets, non-human primates and human are included.

As used herein, the term “amount” refers to “an amount effective” or “an effective amount” of a cell to achieve a beneficial or desired prophylactic or therapeutic result, including clinical results.

As used herein, “therapeutically effective amount” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g., a symptom associated with the disease or condition being treated. As such, “treatment” also includes situations where the disease, disorder, or pathological condition, or at least symptoms associated therewith, are completely inhibited (e.g., prevented from happening) or stopped (e.g., terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent. In some embodiments, the engineered cell or pharmaceutical composition comprising said engineered cell of the disclosure is administered in a prophylactically effective amount.

Embodiments of the present invention provide an apparatus or device as claimed herein and method employing said apparatus or device as claimed herein, where a sample of urine is automatically centrifuged, then an aliquot of the sediment is automatically stained with the conventional Papanicolaou method and then it is visualized by bright field microscopy; and the so stained sample's cellular components are analyzed by Artificial Intelligence (AI) based algorithms using deep learning methods. If based on the automated examination and analysis, suspicion of cancer or identification of otherwise non-identified nucleated cells which are irregular and suspected of forming the basis for cancer risk is made, the so identified sample photos which were captured via automation are automatically sent to a trained pathologist for further examination for suspicion of cancer risk for cancer cells and types that are usually identified by such pathologists for further examination and report preparation, whereas if no abnormal cells are detected/identified, a written report is automatically generated and issued as a negation result for cancer risk which expedites the overall process decreasing overall load of samples to be examined, assessed and reports to be prepared by the pathologist to only the ones suspected of cancer risk, and hence, the method as claimed herein makes it efficient to overcome the problems of false positives and false negatives.

As stated above, there remains a problem and need in the field of conventional and routine urinalysis (UA) for efficiency, standardization, lesser human intervention to overcome issues of false positives and false negatives, and for means and methods that would complement the findings of the UA and lead to identification of anomalies, abnormal and irregular cells that raise concerns and suspicion for cancer risk in a urine sample, while also reporting negation of any such suspicion of cancer risk at this initial diagnosis stage itself without involvement of a pathologist. The present invention as disclosed herein accomplishes that and provides a solution to the aforementioned problems and need by performing the cytological examination procedure automatically, at low cost, in a large number of samples, and without intervention of an expert examiner pathologist. The apparatus and methods as disclosed in the present invention fulfill this by automatically preparing sample slides by centrifuging urine samples onto such slides, and staining said sample slides automatically with the conventional Papanicolaou method, hematoxylin-eosin stain, which is still essential for cancer diagnosis (27-28), this way a large number of samples can be simultaneously and automatically examined at a low cost with the help of automation and AI without any human intervention which is a technical achievement and highly advantageous towards creating a standardized, reliable, reproducible method as disclosed in the present invention. Thus, the apparatus and method as disclosed herein will standardize the performance of the examination and reproducibility of the results by using the same technique to prepare the samples and the same examining methods without human error based in the efficiency of AI. This will also allow comparing current results with previous results and monitoring the evolution of the disease process in an error and bias free manner.

Finally, the method as disclosed herein can automatically detect and recognize abnormal and irregular cells without the intervention of a pathologist to identify samples to be examined further based on cancer risk suspicion which would be categorized and identified separately from samples having normal cells to be identified as negative for suspected cancer risk at this very stage by automated and AI based method as disclosed herein without any human intervention overall reducing the load and burden of samples to be examined and assessed by expert human examiners increasing the overall diagnosis efficiency and reducing the overall time and cost of the UA. In other words, the presently disclosed means and methods concerns only a screening test utilizing automation and AI without human intervention prior to further cancer identification and diagnosis that may be done by any means including conventional clinical pathology processes, and it should not be confused with disclosure of a histopathology procedure because the images of abnormal cells identified in the sediment will be automatically forwarded to a pathologist for accurate and final diagnosis. An advantage of the method as disclosed herein is that by referring the “abnormal cells” to pathologists the method leads to a decrease in the risk of false negatives, which is necessary because there are cases in which the cells may resemble abnormality because of the presence of some cytological features of low-grade urothelial neoplasms but are negative for high grade urothelial carcinoma (HGUC). Another advantage of the method and system as disclosed in the present invention is that it would continue to be improved via machine learning process inherent to the training the AI increasing efficiency and precision of the overall process as time goes on and at the same time the number of such cancer risk suspected cases being referred to a pathologist would decrease leading to a lowering of burden on the experts thereby increasing their precision and efficiency as well.

In an embodiment of the present invention, it provides a method of screening for cancer risk suspicion in a subject undergoing urine analysis or urinalysis referred to as UA, the method comprising the steps of: a) employing an apparatus comprising a mechanical arm with a pipette to automatically suction a fixed volume of unspun urine sample from a collection container, optimally 30 mL, and pour it into a centrifuge tube and insert the centrifuge tube with the urine sample into one of the receptacles of a centrifuge rotating head, said step optionally is performed manually; b) activating by a first button labelled start that is located on the surface of the apparatus to activate the mechanical arm when pressed; c) activating by a second button labelled a print-label that is located on the surface of the apparatus to automatically print a label with each subject identification on each centrifuge tube with the urine sample or optionally manually write on the tube, wherein the printing is done before the tube is inserted in a centrifuge rotating head and while the tubes are held by the mechanical arm, wherein the information to be printed is typed in by an attached keyboard of a first computer and printed on each centrifuge tube with the urine sample when pressing the print-label button or optionally manually written, and optionally, a printed or hand-written label is attached manually to the centrifuge tube with the urine sample; d) transferring the centrifuge tube with the urine sample into a centrifuge automatically, the centrifuge comprising: (i) a rotating head with multiple receptacles to house 12-48 centrifuge tubes, wherein each receptacle accommodates tubes of different sizes, including 10 mL, 30 mL and 50 mL, preferably 30 mL, and (ii) electro-mechanical components to allow different speeds, rotation per minute (rpm) and duration of centrifugation, wherein the centrifuge is used to separate blood and urine; e) centrifuging the centrifuge tube with the urine sample automatically, wherein the centrifuge automatically closes the lid, starts and stops at pre-set rpms which is 1500 rpm for a duration of centrifugation of 5 minutes, and then automatically opens the lid after centrifugation is completed, wherein step e is started by pressing a third button labelled start-centrifuge located on the surface of the apparatus, and optionally, all the steps c to e are automatically run without having to press the print-label and start-centrifuge buttons; f) taking out the centrifuge tube with the urine sample after centrifugation in step e is completed automatically by the mechanical arm of step a or by a different mechanical arm, tilting the tube to pour out the supernatant and tilting the tube back to upright position to keep the urine sediment inside the centrifuge tube, wherein step f starts automatically after the centrifuge stops and the lid opens in step e; g) injecting into the centrifuge tube, a small and fixed volume of saline or other liquid automatically, which is 0.1 milliliter (mL) by a pipette attached to the mechanical arm of step a or a different mechanical arm to re-dissolve the sediment left at the bottom of the centrifuge tube in step f to obtain a re-dissolved sediment in the centrifuge tube; h) suctioning a fixed volume of the re-dissolved sediment in the centrifuge tube and depositing it on a glass slide automatically; i) printing by a printing component on the glass slide of step h automatically, a label with the subject identification, wherein this information is printed by pressing a fourth button labelled print-slide-label located on the surface of the apparatus or optionally by a sent key on the keyboard of the first computer, and optionally, this step is done automatically after the step h without having to type and press the print-slide-label button; j) moving the glass slide labelled in step i automatically by the mechanical arm of step a or by a different mechanical arm or by a conveyor belt to a site for staining the sediment deposited on the glass slide; k) staining the glass slide with the Papanicolaou method at the site for staining automatically either by dipping the glass slide into basins with several fluids or dyes, or by a pipette suctioning these fluids or dyes from the basin and pouring them on top of the slide, wherein the staining procedure in this step follows nine sequential steps including: (1) automatically dipping the glass slide into an acetic acid basin by the mechanical arm of step a or by a different mechanical arm, or optionally the mechanical arm of step a or by a different mechanical arm using a pipette to automatically suction acetic acid from a container and pouring it on top of the glass slide; (2) dipping the glass slide by the mechanical arm of step a or by a different mechanical arm into a water basin for rinsing, or optionally the mechanical arm of step a or by a different mechanical arm using a pipette to automatically suction water from the basin and pour it on top of the slide for rinsing; (3) dipping the glass slide by the mechanical arm of step a or by a different mechanical arm into a water basin for rinsing or optionally the mechanical arm of step a or by a different mechanical arm using a pipette to automatically suction water from the basin and pour it on top of the glass slide for rinsing, (4) dipping the glass slide by the mechanical arm of step a or by a different mechanical arm into an acetic acid basin or optionally the mechanical arm of step a or by a different mechanical arm using a pipette to automatically suction acetic acid and pour it on top of the glass slide; (5) dipping the glass slide by the mechanical arm of step a or by a different mechanical arm into a OG-6 (dye) basin or optionally the mechanical arm of step a or by a different mechanical arm using a pipette to automatically suction this dye from a basin and pour it on top of the glass slide; (6) dipping the glass slide by the mechanical arm of step a or by a different mechanical arm into a EA-50 (dye) basin or optionally the mechanical arm of step a or by a different mechanical arm using a pipette to automatically suction this dye from a basin and pour it on top of the glass slide; (7) dipping the glass slide by the mechanical arm of step a or by a different mechanical arm into a methanol basin, or optionally the mechanical arm of step a or by a different mechanical arm using a pipette to automatically suction methanol from a basin and pour it on top of the glass slide; (8) dipping the glass slide by the mechanical arm of step a or by a different mechanical arm into a xylene basin or optionally the mechanical arm of step a or by a different mechanical arm using a pipette to automatically suction xylene and pour it on top of the glass slide; (9) dipping the glass slide by the mechanical arm of step a or by a different mechanical arm into a tap water basin or by a different mechanical arm using a pipette to automatically suction tap water and pour it on top of the glass slide for rinsing, and optionally, other stains or staining methods are used, including Papanicolaou standard or modified, the Sternheimer-Malbinz method, which is Crystal-violet and safranin with or without toluidine, 34, or Hemacolor, which is Merck, Darnmastadt, Germany, 23, or other sample preparation methods are used including, ThinPrep and centrifugation methods, including Cytospin or the sediment is re-suspended in Cytorich Clear, which is Becton Dickinson Co., Franklin Lakes, NJ, and processed on the SurePath automated system, which is from Becton Dickinson Co. to yield a single, Papanicolaou-stained slide with a stained sediment for visualization of all cells, and optionally, the staining of samples is done manually, wherein the slides are manually inserted into the apparatus for cytological examination; I) depositing a cover slip on top of the glass slide of step k with the stained sediment automatically by the mechanical arm of step a or by a different mechanical arm, wherein the cover slip covers the expanse of the stained sediment, and preferably has a surface of 6 mm², and optionally, this step is done manually; m) moving the glass slide with the stained sediment covered by the cover slip from step 1 automatically by the mechanical arm of step a or by a different mechanical arm or by a conveyor belt under a microscope for visualization of all areas covered by the cover slip, wherein the mechanical arm or the conveyor belt moves the glass slide under the microscope automatically or on command to help the visualization by the microscope of all areas of all cells present and stained on the glass slide, wherein the visualization is done under a bright field microscope, and wherein the bright field microscope comprising: (aa) lenses to give a magnification of ×10 (low power field), ×40 (high power field), and lenses to give a larger magnification for better visualization of nucleolus in search for cancer cells, and (bb) a platform where the glass slide is deposited that moves automatically in a pre-arranged manner to allow visualization of different positions covering all the sediment covered areas under the cover slip, wherein in each of the positions, the images of cells present are sent to a second computer for analysis with a photo taken of said positions, wherein the motion of the platform starts immediately after the slide is deposited on it, wherein the glass slide is first scanned under the ×10 magnification objective and next under the ×40 magnification objective, and optionally, the area of the slide covered with the glass cover could be visualized with a grid to be able to identify the areas where abnormalities are noted; n) analyzing the pictures captured from step m under the second computer with a software comprising various algorithms, the second computer comprising content of stored data including: a Cytopathology Index which is a library or database with thousands of images of all cells that are found in the sample, wherein the Cytopathology Index is made after examining thousands of images published in books describing the Paris System, and images in the library of stored slides of urine cytopathology department of a large medical institutions, including the Mayo Clinic, wherein the images are chosen and approved by more than one expert pathologists, uploaded and stored in a unitary controller file to form the Cytopathology Index, wherein the Cytopathology Index has hundreds or thousands of images stored, wherein new images are added and old ones removed periodically, and wherein the books include Atlas of Renal cytology and histopathologic bases by GB Schumann, a book describing the Paris method by DL Rosenthal; o) removing the glass slide with the mechanical arm of step a or by a different mechanical arm or by a conveyor belt under the microscope after the visualization in step m is completed and depositing it in a storage box for any further examination, and optionally, this step is done manually; p) analyzing with an adequacy algorithm “B” for assessing the proper relationship between specimen source, cytological diagnosis, urine volume, urothelial cellularity, and obscuring features, wherein the obscuring features include non-urothelial cells including vaginal contaminants, bacteria, acute inflammation, sperm, and crystal, wherein said obscuring features in higher amounts obscure the findings and characterization of urothelial cells (Chapter 2, page 7, Ref. 48); q) analyzing with an algorithm “C” for automatically taking photos of each visual field and storing them; r) analyzing with an algorithm “D” for automatically counting the number of each cell in all visual fields examined; s) analyzing with an algorithm “E” that automatically uses data processing methods to enter the findings of the present examination into form “A” and stores the data; t) analyzing with an algorithm “F” that automatically uses data processing methods to enter the findings of the present and previous examinations into form “B” and stores the data; u) analyzing with an algorithm “G” that automatically makes decisions and decides how to proceed after the slide examination is completed, wherein the decisions are selected from a group consisting of: (i) when no abnormal cells are seen, send a command to print a report and send it to the referring physician (P+S) reporting negative cancer risk suspicion, and (ii) when abnormal or atypical or unidentified cells are seen, to send a command to send the slides to an expert pathologist for final review (STP) and reporting the same on probability of cancer risk suspicion; v) employing an algorithm “H” to automatically write a report based on the report in step u, wherein the said report includes patient or subject name, date of birth, sex selected from a group consisting of male and female, previous examination history as a yes or no, referral history, detail on type of specimen, wherein the type can be selected from a group consisting of voided, instrumented, and ileal conduit urine, an adequacy statement, wherein the adequacy statement provides information selected from a group consisting of satisfactory for evaluation statement, wherein the statement provides volume, collection type, cellularity, and cytomorphological findings, and unsatisfactory for evaluation statement, wherein the statement provides information providing reasons including small volume and not enough cells, or recognizable cells, hypercellularity because of acute inflammation; and w) sending the report from step v for printing automatically by a printer.

The Paris system for reporting urinary cytology is a standardized, evidence-based reporting system that provides a comprehensive set of terminology and a diagnostic standard to classify urothelial cells that can be used in either voided or instrumented specimens and for specimens sampled from the lower and upper urinary tract. It was developed to standardize reporting, facilitating communication among pathologists and between pathologists and clinicians. The guiding principle of the system is the detection of HGUC. It increases the sensitivity of diagnosis of high-grade urothelial carcinoma by reducing the rate of indeterminate atypical diagnosis but there is interobserver variability of findings. It comprises seven diagnostic categories: negative for epithelial cell abnormality), negative for high-grade urothelial carcinoma, atypical urothelial cells, suspicious for high-grade urothelial carcinoma, low-grade urothelial neoplasm and other malignancies. The Paris system also support the set of ancillary techniques for indeterminate interpretations.

The Paris system is based on the visually observable or morphological characteristics of the cells, such as size, shape, opacity, geometric complexity, other morphological criteria of malignancy such as enlarged polymorphous cells with prominent lobulated nuclei, high nucleus to cytoplasm ratio, nuclear hyperchromasia, irregular nuclear membranes, irregular, coarse and clumped chromatin. The Paris system uses the Papanicolaou stain, which is polychromatic and includes hematoxylin (stains nucleus dark blue), OG-6 staining solution containing Orange G, and a third staining solution with Eosin Y, Light Green SF yellowish, and Bismark brown. Eosin Y is pink and stains the cytoplasm of epithelial squamous cells cytoplasm orange to pink. Large medical institutions have hundreds of slides stored from examinations done over many years (49).

The present invention utilizes AI and stored data, which had been manually entered and can be used to help in the diagnosis, including: (a) bulk image data containing confirmed categories of atypical cells, (b) actuarial tables relating to individual patient risk profile (age, sex, exposure to carcinogens), (c) relevant medical history such as history of cancer, exposure to chemicals linked to bladder cancer, such as arsenic and chemicals used in the manufacture of dyes, rubber, leather, textiles and paint products, (d) previous cancer treatment, and (e) treatment with the anti-cancer drug cyclophosphamide (these increase the risk of bladder cancer), which are used to train the AI in machine learning so as to train and automate the system and method to formulate the Cytopathologic Index of the present invention as disclosed herein. The Cytopathologic Index also has images of other cells present in the sediment, i.e. non-nucleated cells (red cells), and leucocytes used to train the AI for apparatus, system and method of the present invention as disclosed so as to train it to distinguish and characterize such various categories of cells to be applied for detecting and diagnosing the suspicion for cancer risk or negating the same at this stage itself before it goes for further characterization and confirmation outside of the AI-driven automation of the present invention.

In the present invention there are two kinds of stored banks of forms, namely, form A and form B. Form “A” or table “A” is a list of the names of all the diagnostic categories inclusive of types of cells identified based on the Paris System for reporting urinary cytology that could be present, and the other non-urothelial cells that can be present. All types and number of cells in the current examination are entered in the form A. Form “B” or table “B” is like table “A” but has several columns with results and narratives of the current examination and previous examinations to facilitate comparison of results obtained over time.

The present invention provides an apparatus, system and method using them for imaging and analysis steps of the method as disclosed herein, where, an algorithm “A” is employed to visualize and identify all cells in a slide under high and low power of magnification under the microscope, especially the urothelial cells using the same basis as the Paris system and other established diagnostic criteria which are traditionally and routinely used to choose the cells included in the Cytopathologic Index of the present invention as described herein above. A unique, critical, and advantageous aspect of the method as disclosed in the present invention for assessing the susceptibility of cancer risk is to use the machine trained AI for automated detection and identification of cells imaged and captured using the microscope and comparing the captured images seen in the slide with the stored images in the Cytopathology Index in the unitary controller used for identifying and characterizing the cells in the captured images for automated positive or negative identification of susceptibility for cancer risk based on UA which can then form the basis of report for a subject for negative cancer risk or can form the basis for further downstream examination and confirmation by a trained pathologist for instance of the positive susceptibility for cancer risk. In the present invention, in the method as disclosed herein and as has been previously mentioned, the cells are identified based on characteristics that are automatically compared with stored images to assess and analyze images captured in the present method, including, cytoplasm color, shape, contrast, size, texture, the presence and type of cell membrane, the nucleus color, size, shape, number of nuclei, texture, coarseness of chromatin, and size, shape, and number of nucleoli.

Using the present invention, in the method disclosed herein, following cells are identified, including: (a) urothelial cells: non-diagnostic (or unsatisfactory), negative for high-grade urothelial carcinoma, atypical cells (abnormal but not cancer cells), suspicious for high-grade urothelial carcinoma (likely cancer cells), low grade urothelial neoplasm, and other malignancies; (b) other malignancies can includes: (a) squamous cells carcinoma, adenocarcinoma, and small-cell carcinoma of the bladder, (b) secondary malignancies in the bladder by direct invasion, i.e. melanoma (35-36), lymphoma (37-38), renal cell carcinoma, prostatic carcinoma; (c) other identifiable nucleated cells: lymphocytes, monocytes, neutrophils, eosinophils, renal tubule epithelial cells; (d) non-nucleated cells such as red blood cells; (e) unidentified nucleated cells, wherein malignant non-urothelial cells could be reported as unidentified nucleated cells; (f) non-cellular sediment which include crystals, debris, etc.; (g) other types of cells that can be found in UA such as bacterial organisms, fungal organisms, viral components such as CMV, herpes, adenovirus, polyomavirus, etc.

Even though the purpose of the device is to identify urothelial cells, the software as disclosed and used in the present invention also identifies other solid components in the sediment as automatically formed in the steps of the method of the present invention as disclosed herein, that include, debris, casts, crystals, since the images stored in the Cytopathologic Index of the present invention including other solid components, and then the automated machine learning trained AI is used to (b) reject cells as “non-urothelial” or abnormal or irregular cells for identification and detection of susceptibility for cancer risk comprising the steps of first thoroughly examining and identifying such cells, and after identifying such cells based on their characteristics as described herein above, then said identified characteristics are matched with images in the Cytopathologic Index to automatically decipher negative or positive susceptibility for cancer risk and either prepare a report for negative risk based on just automated, non-human AI based method of the present invention as disclosed herein, or to only forward the slides and data for further examination, characterization, and analysis with human intervention for instance by a trained pathologist.

As used herein, the Paris System identifies various diagnostic categories of cells based on their overall characteristics for each of said categories which are as described below and information for the said description and photos corresponding to said categories are incorporated by reference (48-49). Category #1: Negative for epithelial cell abnormality. Category #2: Negative for High Grade Urothelial Carcinoma (NHGUC): To minimize lumping everything into the “atypical” category, the terminology Negative for High Grade Urothelial Carcinoma includes all entities that pose no significant risk to the patient for developing HGUC based upon available studies. This term also clarifies the goal of the Paris System—to highlight those cases at risk for HGUC. For example, radiation associated atypia is classified as Negative for High Grade Urothelial Carcinoma and not atypical. A urine sample (voided or instrumented) is considered Negative for High Grade Urothelial Carcinoma if any of the following benign cytologic changes are present: (i) Benign urothelial, squamous and glandular cells. (ii) True tissue fragments and clusters without morphologic changes. (iii) Benign urothelial tissue fragments. (iv) Changes associated with stones. (v) Viral cytopathic effect due to polyoma virus. (vi) Post-treatment effect of bladder instillations, especially BCG. (vii) Post-therapy effect, for non-bladder disease, e.g., pelvic irradiation for other malignancies, systemic chemotherapy that may affect the urothelium i.e., Cyclophosphamide. (viii) Enteric epithelium following a surgical urinary diversion post-hysterectomy, including epithelial cells from urinary diversions. (ix) Unexpected normal cells, e.g. sperm, seminal vesicle cells, cells from the female genital tract. (x) These are urothelial cells with morphology changes with non-neoplastic phenotype changes. Negative: include urothelial cells with morphology changes with non-neoplastic phenotypic changes (48).

Category #3: Benign/reactive superficial (umbrella, cap or dome) cells have a very frothy and abundant cytoplasm resulting in a low nucleus/cytoplasm ratio. Nuclei have pale finely granular chromatin. Nucleoli can be prominent, but do not reflect any abnormality. Multinucleation is common, especially in instrumented samples.

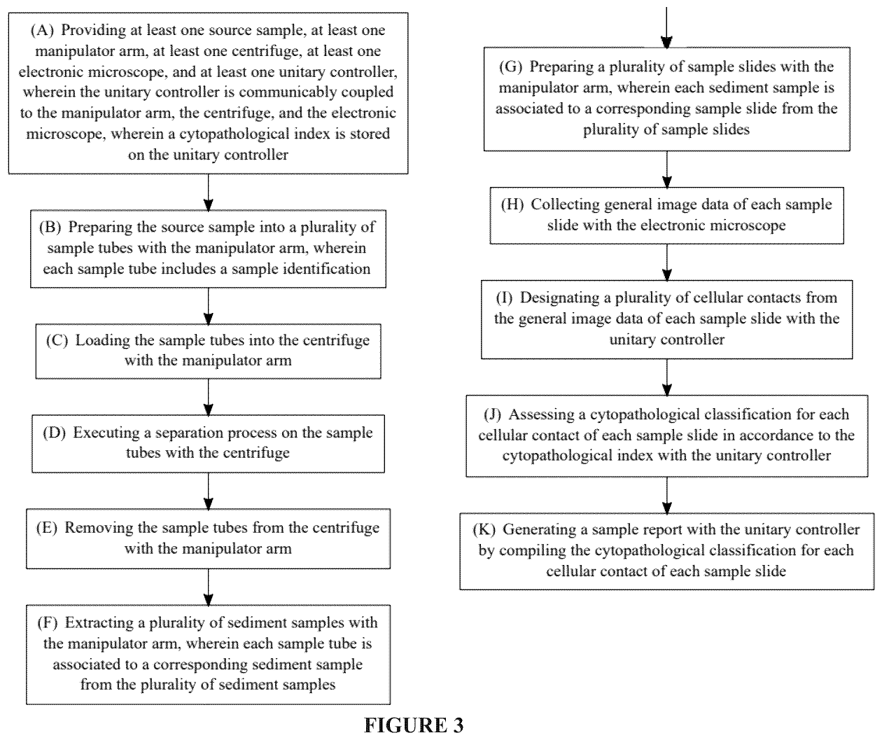

Category #4: Superficial cells also can be present in clusters of smaller cells. The nuclei of these cells are darker and slightly smaller than in superficial cells but the nuclear shapes are round, nuclear membranes are smooth and architecture is uniform. Nucleus to Cytoplasm (N/C) ratios are high due to the small amount of cytoplasm of each cell. These cells are the most superficial cells in the bladder, creating an “Umbrella” over all urothelial cells. Their nuclear and cytoplasmic character is the same as other superficial cells, but additionally, they possess a thickened cytoplasmic edge that does not go all around the cell. These cells are shown in FIG. 3.1 in Ref. 48. Superficial cells may appear very atypical by virtues of enlarged nuclei and multiple nucleoli, they are recognized as benign/reactive by their low N/C ration, characteristic scalloped edges, vacuolated cytoplasm, and smooth nuclear membranes. These cells are shown in FIG. 3.3, a and b, in Ref. 48.

Category #5: Superficial urothelial cells are large, shaped like the canopy of an umbrella, with rounded convex luminal surfaces and scalloped (concaved) borders onto which the underlying intermediate ells are sometimes attached. The cytoplasm is abundant and vacuolated or foamy, not to be mistaken as koilocytes. They are often bi- or multinucleated or may contain a single large nucleus. The nuclei are centrally located, round to oval, with smooth nuclear membranes. The chromatic is fine, and an occasionally prominent chromocenter/nucleolus is present. Characteristically, the nuclear/cytoplasm ratio is low. These cells are shown in FIG. 3.1, page 14 in Ref. 48.

Category #6: Intermediate urothelial cells have nuclei with basically the same size and character as superficial cells, but have less cytoplasm, imparting a higher N/C ratio. Since they are less mature than superficial cells, their nuclear chromatic may be a bit coarser than the ubiquitous superficial cells, thin, even nuclear membranes and uniform chromatin distribution will support their totally benign condition. Cytoplasm will not be as vacuolated as superficial cells, but is not completely opaque (homogenous), the latter feature being cited as a characteristic of low-grade urothelial neoplasms. These cells are shown in FIG. 3.2 page 16 in Ref 48.

The above-described normal cells must be differentiated from other normal cells that can be present in the sediment including: Category #7: Squamous epithelial cells (from urethra, or contaminant from the vagina or perineum. These cells are shown in FIG. 3.4 page 18 of Ref. 48. Category #8: Glandular cells (from cervix or endometrium) are small, have scant cytoplasm, slightly irregular nuclei with vesicular fine chromatin, and visible small nucleoli. These cells are of interest in post-menopausal women. They are shown in FIG. 3.5, page 19 in Ref. 48. Category #9: Renal tubular epithelial cells usually appear degenerated and resemble histiocytes. They are shown in FIG. 3.7 a-c page 19 in Ref. 48. Category #10: Urothelial cells with changed associated with lithiasis, viral cytopathic effect (FIG. 3.12, page 28, in Ref. 48), and post-therapy with BCG effect (FIG. 3.13, page 31, in Ref. 48), in bladder diversion urine (FIG. 3.15a, page 33, in Ref. 48). Chemotherapy: Intravesical mitomycin and Thiotepa usually affect superficial cells and cause nuclear enlargement, multinucleation and hyperchromasia of those cells, all worrisome but non-specific. Systemic cyclophosphamide has been associated with urothelial hyperchromasia and degeneration, plus the presence of large nuclei and increased N/C ratios.

Category #11: Atypical Urothelial Cells (AUC): Defined as cellular changes that fulfill the major (required) criterion and only 1 minor criterion. The presence of 2 or more minor criterion including nuclear hyperchromasia is diagnostic of Suspicious for HGUC (see below). Major criterion (required): Non superficial and non-degenerated urothelial cells with an increased N/C ratio (>0.5). Minor criteria (one required): (i) Nuclear hyperchromasia. (ii) Irregular nuclear membranes. (iii) Irregular, coarse and clumped chromatin. (iv) Diagnosis of AUC is appropriate when cells are more abnormal than NHGUC. (v) AUC is appropriate when there is suspicion of HGUC but also extensive degeneration. Normal intermediate and basal urothelial cells, typically seen in instrumented urine, have high N/C ratio and frequently occur in groups; should be regarded as NHGUC.

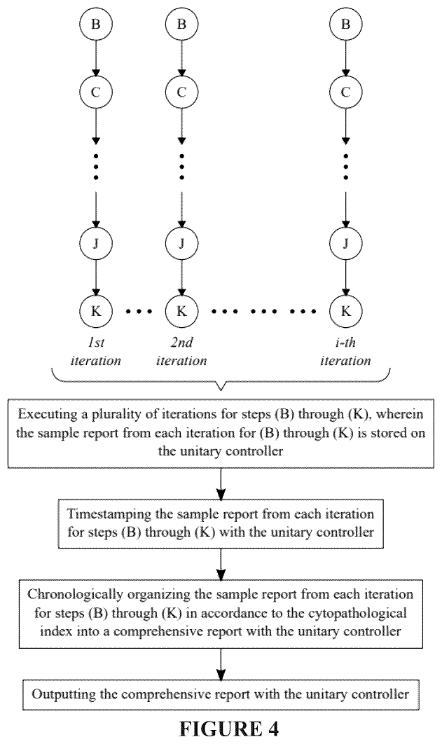

The above-introduced AUC are cells with mild to moderate cytologic (not architectural) atypia. These cells have cytologic changes that fall short of a diagnosis of suspicious for High-grade urothelial carcinomas or high-grade urothelial carcinoma and requires exclusion of changes in which the reason for the “atypia” is known, such as changes caused by polyoma virus and other infections, reactive umbrella cells, seminal vesicle cells, and reactive changes due to stones, instrumentation, and therapy. Normal benign/reactive urothelial cells (shown in FIGS. 4.1 and 4.2, pages 40-41 in Ref. 48) can be compared with AUC (shown in FIGS. 4.3, 4.4, 4.5, 4.6, 4.7, 4.8, 4.9, 4.10 shown in pages 41-45, in Ref. 48).

The above-mentioned AUC is defined as cellular changes that fulfill the major criterion and at least one minor criterion or the presence of two or more minor criteria unless there are marked degenerative changes: major criterion (required): non-superficial and non-degenerated urothelial cells with an increased nuclear cytoplasm (N/C) ratio (>0.5) (explanatory note 1). Minor criteria (one required): (i) Nuclear hyperchromasia (explanatory Note 2). (ii) Irregular nuclear membranes (chromatinic rim or nuclear contours) (explanatory Note 3). (iii) Irregular, coarse, clumped chromatin. Diagnosis of AUC is appropriate when cells are more abnormal than NHGUC. AUC is appropriate when there is suspicion of HGUC but also extensive degeneration. Normal intermediate and basal urothelial cells, typically observed in instrumented urine specimens, should be identified and categorized as “normal” or NHGUC despite the fact that they have a high N/C ratio and may appear mildly hyperchromatic (FIG. 4.1, page 20 in Ref. 48). These cells frequently occur in groups, show uniform, round nuclei and inconspicuous nucleoli with finely dispersed, smooth chromatin. Explanatory Note 1: High N/C ratio. HGUC cells often show a high N/C ratio exceeding 0.7 (meaning 70% of the area of the cell is occupied by the nucleus). For a diagnosis of AUC, the N/C ratio should be at least 0.5 (50%). If this is the sole finding, the case should not be reported under the AUC category.

Explanatory Note 2: Nuclear hyperchromasia. Hyperchromasia refers to an increased density of the nuclear chromatin of urothelial cells as compared with that of normal superficial urothelial cells (preferably) or intermediate squamous cells. Hyperchromasia reflects increased light absorption because of increased chromatin density and affinity for nuclear dyes, variably seen in neoplastic cells.

The staining intensity and texture of the nuclear chromatin should not be so pronounced as that of cells in the SHGUC or HGUC categories. Explanatory Not 3: Irregular nuclear membrane. Compared with the round shape and smooth contours of the nuclei of normal urothelial cells, AUC usually show and irregular nuclear shape and variably thickened chromatin rim while sill retaining a generally round, not oval, shape.

Other features that may be present in AUC: (i) Eccentric nuclei, in cells without columnar features are usually a sign of loss of nuclear polarity: Urothelial cells with eccentric nuclear and high N/C ratios may raise the suspicion of malignancy. The differential diagnosis of sch cells with eccentric nuclei includes native type of glandular cells (cystitis glandularis) and reactive renal tubular cells, which lack hyperchromasia, nuclear membrane irregularity, and irregular clumped chromatin. Cases in which eccentric nuclei are the sole finding should not be reported as AUC. (ii) Presence of urothelial cell clusters in voided urine specimens: The mere presence of benign clusters in voided urine specimens does not fulfill the criteria for AUC, unless the urothelial cells within the group also show two to the described cytologic criteria (one major and one minor). (iii) Large nuclear size: The nuclei of AUC cells are usually larger than that of intermediate or basal urothelial cells, intermediate squamous cells, or benign columnar cells. However, decrease or normal appearing nuclear size can be associated with cellular shrinkage and may occasionally be seen in cells otherwise fulfilling the diagnostic criteria for AUC.

Category #12: Suspicious for High Grade Urothelial Carcinoma (SHGUC): (i) Suspicious for HGUC or “SHGUC” means to reflect the presence of urothelial cells with severe atypia that falls short for a diagnosis of high-grade urothelial carcinoma (HGUC), but beyond atypia that is associated with the atypical urothelial cells (AUC) category. (ii) This diagnosis is restrictively used in cases with abnormal urothelial cells that quantitatively fall short of a definitive diagnosis of HGUC. Criteria: (a) A diagnosis of SHGUC is defined as non-superficial and non-degenerated urothelial cells showing: Increased N/C ratio, at least 0.5-0.7 (required criterion). (b) Moderate to severe nuclear hyperchromasia (required criterion) and at least one of the following: (1) Irregular clumpy chromatin. (2) Marked irregular nuclear membranes. Examples of SHGUC are shown in figures or Fig (used interchangeably throughout this disclosure) 5.1, 5.2, 5.3 5.4, 5.5, 5.6, 5.7, 5.8, 5.9 in pages 50-54 in Ref. 48.

The present invention in its method as employed and disclosed herein, using all the features listed above, the decision to assign the case of a subject's UA into the “SHGUC” or the “positive for HGUC” categories is based on the number of the abnormal cells fulfilling the above criteria. In general, it is accepted that (a) if there is a minimum of five abnormal, well preserved of these cells present should be categorized as SHGUC. In instrumented specimens a minimum of ten of these cells is required for a diagnosis of positive for High-grade UGUC.

As used herein, hyperchromasia refers to an increased density of the nuclear chromatin of abnormal urothelial cells. A mild difference in chromasia between the cells in question and the umbrella or intermediate and the accompanying cells is not diagnostic.

Category #13: High Grade Urothelial Carcinoma (HGUC): (i) Sensitivity of urine cytology for HGUC is 50-85%. (ii) Positive urine cytology is clinically meaningful, is significantly associated with tumor recurrence and is independent of other clinicopathologic variables. Hence, positive urine cytology in primary upper urinary tract urothelial carcinoma is valuable to predict prognosis and preoperative positive urine cytology may be associated with higher prevalence of tumor recurrence. (iii) It can be useful to predict tumor progression. But it is to be noted that: (a) urine cytology cannot distinguish invasive HGUC and carcinoma in Situ (CIS). (b) Squamous or glandular differentiation of urothelial carcinoma may be seen in urine cytology but a diagnosis of squamous cell carcinoma or adenocarcinoma of the urinary tract can only be made after examination of biopsy or cystectomy specimens. (c) HGUC is diagnosed on the basis of this criteria according to the Paris System consensus: (1) Cellularity; at least 5-10 abnormal cells. (2) N/C ratio: 0.7 or greater. (3) Nucleus: moderate to severe hyperchromasia. (4) Nuclear membrane: markedly irregular. (5) Chromatin: coarse/clumped. (d) Other notable cytomorphologic features of HGUC are: (1) Cellular pleomorphism. (2) Marked variation in cellular size and shapes. i.e. oval, rounded, elongated or plasmacytoid (comet cells). (3) Scant, pale or dense cytoplasm. (4) Prominent nucleoli. (5) Mitoses. (6) Necrotic debris is and indicator of invasive disease. (7) Inflammation. Examples of HGUC are shown in FIG. 6.1, 6.2, 6.3, 6.4, 6.5, 6.6, 6.7, 6.8, 6.9, 6.10, 6.11, in Ref. 48.

Category #14: Low Grade Urothelial Neoplasia (LGUN): (i) LGUN is a combined cytologic term for low grade papillary urothelial neoplasms, which includes urothelial papilloma, papillary urothelial neoplasm of uncertain malignant potential (PUNLMP) and low grade papillary urothelial carcinoma (LGPUC). (ii) Definitive diagnosis of LGUN is possible only in the presence of this cytologic criteria (regardless of voided urine or instrumented urine): (a) 3 dimensional cellular papillary clusters with fibrovascular cores including capillaries (shown in FIG. 7.1, 7.2, 7.3 in Ref. 48). (b) Cellular papillary clusters are defined as clusters of cells with nuclear overlapping forming papillae. (iii) The following cytologic features should be categorized as NHGUC: (a) Three dimensional cellular clusters without fibrovascular cores (shown in FIG. 7.5, page 81, in Ref. 48). (c) Increased numbers of single monotonous (non-umbrella) cells (shown in FIG. 7.5b in Ref. 48). (iv) Cytoplasmic homogeneity. (v) Nuclear border irregularity. (vi) Increased N/C ratio. (vi) LGUN in these cases without definitive cytomorphologic features. (vii) Rate of progression is 0% for papilloma, 3.6% for PUNLMP and 5-25% for LGPUC (WHO/ISUP classification (2004)).

Besides the presence of the following features reported as characteristic for LGPUC may also be associated with HGUC: (a) Cytoplasmic homogeneity (shown in FIG. 7.6, page 82 in Ref. 48). (b) Nuclear border irregularity (shown in FIG. 7.7, page 82, Ref. 48). (c) Increased nuclear/cytoplasmic ratio.

Other malignancies primary and metastatic categories include: Category #15: Squamous Cell Carcinoma (SCC): (i) Accounts for 2-5% of all bladder cancer in West. (ii) In North Africa and Middle East where Schistosoma haematobium infestation is endemic, it accounts for 25-30% of bladder malignancies. (iii) Cases not associated with Schistosoma (non-bilharzial) are usually associated with conditions causing urinary stasis with epithelial injury, such as spinal cord injury or paraplegia. (iv) Cytologic features of bladder SCC are similar to SCC elsewhere. (v) Diagnostic criteria for SCC: (a) Cellular specimen with numerous individual and nests of squamous cells (seen in FIGS. 8.2 and 8.3, in Ref. 48). (b) Tumor cells are large, polygonal with keratinized cytoplasm, sharp borders and mildly to markedly atypical hyperchromatic nuclei (shown in FIG. 8.3, 8.4, pages 90-91 in Ref. 48). (c) Fiber and tadpole cells, squamous pearls and “cell in cell” arrangement may be present (shown in FIG. 8.2, 8.3, 8.4, 8.5, pages 90-91 in Ref. 48). (d) Background may show fragments of a nucleated squamous cells, small atypical parakeratotic cells, necrosis, RBCs and neutrophils. (e) Nonkeratinizing malignant cell groups with metaplastic appearance may be present (shown in FIG. 8.6, page 92 in Ref. 48). (f) Liquid based preparations show similar morphology but the background is cleaner, so cell details are better preserved.

Category #16: Adenocarcinoma: (i) Accounts for 0.5-2.5% of all primary bladder malignancies and includes vesical and urachal subtypes. (ii) Urachal adenocarcinoma develops within urachal remnants located in bladder dome. (iii) Primary urinary tract adenocarcinoma is less common than secondary involvement from adjacent organs. (iv) Risk factors include cystitis glandularis of intestinal type and bladder exstrophy. (v) Diagnostic criteria: (a) Variable cellularity. (b) Enteric/colonic type columnar cell clusters and single degenerated cells in a background of necrosis and mucin (shown in FIG. 8.9, page 94 in Ref. 48). (c) Nuclei are large vesicular or hyperchromatic, with irregular shapes and prominent nucleoli. (d) Cytoplasm may be vacuolated (shown in FIG. 8.10 in Ref. 48). (vi) Mucinous/colloid type has rounded 3-D clusters of crowded, bland cells with small to moderate amounts of lacy cytoplasm with occasional mucin vacuoles and medium sized nuclei with visible nucleoli in mucinous background. (vii) Signet ring cells carcinoma displays cells with a large cytoplasmic containing vacuole that may appear optically clear or finely vacuolated and pushes the crescent-shaped hyperchromatic nucleus to the periphery of the cells (shown in FIG. 8.11, page 96, in Ref. 48). (viii) Clear cell carcinoma: cells with abundant vacuolated cytoplasm and centrally located nuclei that may be present in clusters with hobnail configuration. (ix) Clear cell AdCa has cells with abundant vacuole cytoplasm and centrally located nuclei that may be present in clusters with hobnail configuration (shown in FIG. 8.12, page 97, in Ref. 48). (x) Differential diagnosis of well-differentiated primary AdCa of the bladder includes glandular cells from a fistula with the vagina or the gastrointestinal tract, cystitis glandularis, intestinal metaplasia, nephrogenic metaplasia/adenoma, and villous adenoma.

Category #17: Small cell carcinoma: (i) Accounts for less than 1% of all bladder malignancies. (ii) Diagnostic criteria: (a) Moderate to high cellularity. (b) Hemorrhagic and necrotic background with apoptosis, isolated or small groups of small, undifferentiated malignant cells, mitoses and numerous neutrophils. (c) Cells are arranged singly, in linear pattern with rosettes, loosely or tightly cohesive clusters (shown in FIG. 8.15, page 101 in Ref. 48). (d) Tumor cells are round to oval or irregular and small to medium in size (2-3 x lymphocytes). (e) Nuclei are small to oval, hyperchromatic with finely granular evenly distributed or smudged chromatin, ill-defined membranes, prominent molding and display crush artifact. (f) Nucleoli are inconspicuous or absent (shown in FIG. 8. 16, page 101 of Ref. 48). (g) Scanty cytoplasm. (h) High N: C ratio. The cytological differential diagnosis includes metastatic SMCC from the lung, poorly differentiated small cell type SqCC, carcinoid, UC, lymphoma, melanoma ad other small cells malignancies.

Category #18: Secondary neoplasms: Category #18A: Renal cell carcinoma: (i) May be seen in cases of renal pelvis invasion by renal cell carcinoma. (ii) Degenerated cells. (iii) Cells maintain the same morphology as the tumors in the kidney. Category #18B: Prostatic carcinoma: (i) Bladder neck involvement by prostatic carcinoma. (ii) Large cells in clusters with ill-defined cell borders, vacuolated cytoplasm, round nuclei and prominent nucleoli. (iii) Immunohistochemistry may be helpful. Category #18C: Colonic carcinoma: (i) Direct extension to bladder with possible fistulae formation. (ii) Cells arranged in acinar configuration. (iii) Coarse chromatin pattern and prominent nucleoli. (iv) Necrosis and abundant red blood cells. (v) Fecal material present.

As used herein, the Cytopathological Index of the present invention includes all photos that have been and will be provided by cytopathological departments that collaborate in developing said Cytopathology Index for training the AI and developing the method, apparatus, and system as disclosed in the present invention. The cytopathology departments of any large medical institution have a library with hundreds or thousands of slides and their captured images showing all the cells described above, which are used and will keep being used for machine learning and training of AI.

In the present invention and the method as disclosed herein, an adequacy algorithm “B” for the proper relationship between specimen source, cytological diagnosis, urine volume, urothelial cellularity, and obscuring features. Obscuring features include non-urothelial cells such as vaginal contaminants, bacteria, acute inflammation, sperm, and crystal which, when copious may obscure the findings and characterization of urothelial cells (refer Chapter 2, page 7, Ref. 48). This algorithm is similar to the one commonly in use and described in Rosenthal on page 7. A description of the algorithm in prose is: “If an atypical cell is present: report as adequate specimen; if absent and samples was Instrumented: report as Appropriate Benign Urothelial cellularity; if sample was not instrumented and there are non-urothelial features obscuring urothelial morphology: report as inadequate specimen; if there are not non-urothelial features obscuring urothelial morphology: report as appropriate benign urothelial cellularity, if the urine sample volume is adequate (>30 mL), but report as inadequate specimen, if the urine sample volume is inadequate (<30 mL)” (refer page 7, Ref. 48).

As used herein, the adequacy statement provides: a) general categorization: negative for epithelial cell abnormality, epithelial cell abnormality present; b) descriptive diagnosis: bacterial organisms, fungal organisms, viral changes (CMV, herpes, adenovirus, polyomavirus), nonspecific inflammatory changes, acute inflammation, chronic inflammation, changes consistent with xanthogranulomatous pyelonephritis, cellular changes associated with chemotherapeutic agents, or radiation, epithelial cell abnormalities: atypical urothelial cells, low-grade urothelial carcinoma, high-grade urothelial carcinoma, squamous cell carcinoma Adenocarcinoma, and other malignant neoplasms (specify type); c) other information: optional comment section (Pansare V, Pathology Outlines.com, Inc, November 2021) that includes any other data has been entered manually.

As used herein, the adequacy algorithm is similar to the one commonly in use and described in Rosenthal page 7). A description of the algorithm in prose is: “If an atypical cell is present: report as adequate specimen; if absent and samples was Instrumented: report as Appropriate Benign Urothelial cellularity; if sample was not instrumented and there are non-urothelial features obscuring urothelial morphology: report as inadequate specimen; if there are not non-urothelial features obscuring urothelial morphology: report as appropriate benign urothelial cellularity, if the urine sample volume is adequate (>30 mL), but report as inadequate specimen, if the urine sample volume is inadequate (<30 mL)” (quotes from page 7, Ref. 48).

In the present invention, in the method as disclosed herein, the steps of image analysis employ method steps, where the urothelial cells are analyzed by algorithms as disclosed herein that identify them on basis of color, shape, contrast, size, and texture. The image of each cell seen in a high-power field of the microscope are compared with images of components stored in the software database, or library of stored images in the Cytopathological Index as disclosed in the present invention. The image analysis is preferably done using deep learning methods, including, artificial neural network (ANN, wherein the entire image is fed as an array of numbers), or a variant thereof, including, convolutional neural network (CNN, wherein the image is broken up into a number of tiles) using Wide Res net 50 residual network formulation or other methods available. In these methods that are employed in the present invention as disclosed herein, the data goes through multiple layers of nonlinear processing units for feature extraction and transformation (39-45). Colors are represented as a combination of red, green, blue, and the second computer in the method as disclosed herein above for the present invention interprets images by converting them into an array which is then compared to patterns of numbers against already-know objects (or labeled objects). Pre-training of the deep network with a large set of “labeled” images is necessary to acquire knowledge to extract images from the examined slide. Pre-training includes images of components found in normal and abnormal urine samples that have been labeled, that is, annotated (or validated) by an expert examiner such as a pathologist or renal physician, and obtained from photos in books and other publications and from the expert's own database (46-48, 8). This is also called virtual peer review because images for comparison are extracted from own database.

In the present invention, in the method as disclosed herein, other methods could be used to stain the samples and to analyze the sediment samples, for example, methods that use handcrafted features (well established feature extraction methods such as local binary pattern and encoded local projections give high identification accuracies, or discriminative models could be used to classify data into different groups (this method also needs labeled data)). That is, using deep learning for image recognition, the raw input may be a matrix of pixels; the first representational layer may abstract the pixels and encode edges; the second layer may compose and encode arrangements of edges; the third layer may encode an object, i.e. nucleus, cell or crystal, etc. Deep learning generative methods (which usually focus on learning to re-produce data without making any decisions) probably would not be suitable. And the algorithms will be unsupervised (will scan all the slides, not chosen areas of interest. After all the components of the sediment are identified and labeled, the average number of each type of components in all the fields examined will be calculated.

In the present invention, in the method as disclosed herein, because whole slide imaging (WSI) deals with gigapixel digital images of large size, usually several thousand by several thousand pixels which require specialized graphical processing units (GPUs) with large memory and high speed, to examine the sediment with WSI may require “patching” that is dividing the whole image into many small tiles, i.e. much smaller than 1000×1000 (deep ANNs operate in images of up to 350 by 350 pixels), however fine details could be missed.

In an embodiment of the present invention, it provides an apparatus or a system of screening for cancer risk suspicion in a subject undergoing urinalysis or urine analysis, the apparatus or system comprising: a) a mechanical arm with a pipette to automatically suction a fixed volume of unspun urine from a collection container, preferably a volume of 30 mL, pours it in a centrifuge tube and move and insert the tube in a receptacle in a centrifuge rotating head; b) a “start” button to activate the mechanical arm, located on the surface of the apparatus; c) a printing component to label each centrifuge tube with each patient identification; d) a centrifuge with internal mechanics to automatically close the lid, start and stop at pre-set rotation per minutes referred to as rpms and duration of centrifugation, and open the lid after the centrifugation is completed; e) a “start-centrif” button to start centrifugation; f) the same mechanical arm or a different mechanical arm to automatically take the tubes out of the centrifuge, tilt them to pour out the supernatant and tilt them back to upright position keeping inside the tube the urine sediment; g) the same mechanical arm takes a pipette or a different mechanical arm with a pipette to automatically inject a small and fixed volume of saline, preferably, the fixed volume is 0.1 mL or other liquid into the tube to re-dissolve the sediment left at the bottom of the tube, suctions a fixed volume of re-dissolved sediment from the bottom of each centrifuge tube and deposit it on a glass slide; h) a printing component to label each slide with the patient identification printed in the centrifuge tubes; i) a button “Print-label-slide” located on the surface of the apparatus to initiate printing of the label in the glass slide; j) the same mechanical arm or a different mechanical arm or a conveyor belt to move the glass slide to a site where the sediment is stained; k) the same mechanical arm takes a pipette or a different mechanical arm stain the slide with the Papanicolaou method in four steps by either dipping the slide into different basins containing different fluids or dyes or holding a pipette to suction these fluids/dyes and pour them on top of the slide; I) basins with different fluids/dyes for staining; m) the same or a different mechanical arm to deposit a cover slip on top of the glass slide with stained sediment; n) the same mechanical arm or a different mechanical arm or conveyor belt to move the slide with stained sediment under the microscope; o) a standard bright field microscope with 10× and 40× magnification; p) a computer that has vision capabilities to visualize all the images seen by the microscope, several algorithms, and various narratives that could be used to report the results; q) an algorithm “A” to identify with computer vision all the urothelial cells and other cells; r) an algorithm “B” for adequacy of the sample; s) an algorithm “C” that automatically takes photos of visual fields with abnormal cells; t) an algorithm “D” that count cells in all visual fields examined; u) an algorithm “E” that using data processing methods enters the findings of the present examination into Form “A” and stores the data; v) an algorithm “F” that uses data processing methods to enter the results of current examination and previous examinations into Form “B” to allow comparison of results and stores the data; w) an algorithm “G” makes decisions: no abnormal cells, it sends the populated Form “A” and a narrative to a printer to issue written report and to send it to the referring client, and if abnormal cells are present, it sends the photos with the abnormal cells to a pathologist for final analysis, to send commands to the mechanical arm, centrifuge, microscope, to the printers (to print labels for urine samples, tubes with urine to be centrifuged, slides, to write and print reports, to send data to a pathologist or referring client; x) an algorithm “H” writes reports; and y) a printer that prints the reports prepared by the method providing a result of negative for cancer risk susceptibility or to convey a probability of cancer risk susceptibility and need for further examination for which the slides and report are forwarded to an expert pathologist . . .

In an embodiment of the present invention, it provides a method of screening for cancer risk suspicion in a subject undergoing urinalysis or urine analysis, the method comprising the steps of: a) suctioning automatically a fixed volume, preferably a volume of 30 mL, of unspun urine from a collection container a mechanical arm with a pipette of an apparatus to pour it in a centrifuge tube and move and insert the tube in a receptacle in a centrifuge rotating head; b) activating the mechanical arm by hitting a “start” button located on the surface of the apparatus; c) printing automatically with a printing component to label each centrifuge tube with each subject or patient identification; d) processing automatically with a centrifuge with internal mechanics to automatically close the lid, start and stop at pre-set rotation per minutes referred to as rpms and duration of centrifugation, and open the lid after the centrifugation is completed; e) activating the centrifuge with a “start-centrif” button to start centrifugation, which can be programmed to be activated automatically by a computer; f) moving automatically the tubes using the same mechanical arm or a different mechanical arm to automatically take the tubes out of the centrifuge, tilt them to pour out the supernatant and tilt them back to upright position keeping inside the tube the urine sediment; g) injecting automatically using the same mechanical arm takes a pipette or a different mechanical arm with a pipette to automatically inject a small and fixed volume of saline, preferably, the fixed volume is 0.1 mL or other liquid into the tube to re-dissolve the sediment left at the bottom of the tube, suctions a fixed volume of re-dissolved sediment from the bottom of each centrifuge tube and deposit it on a glass slide; h) labeling using a printing component to label each slide with the patient identification printed in the centrifuge tubes; i) pushing or automatically programming a button “Print-label-slide” to be pushed, which is located on the surface of the apparatus to initiate printing of the label in the glass slide; j) moving using the same mechanical arm or a different mechanical arm or a conveyor belt or automatically programming to move the glass slide to a site where the sediment is stained; k) staining using the same mechanical arm takes a pipette or a different mechanical arm or automatically programming to stain the slide with the Papanicolaou method in four steps by either dipping the slide into different basins containing different fluids or dyes or holding a pipette to suction these fluids/dyes and pour them on top of the slide, wherein basins with different fluids/dyes are used for staining the slide; I) putting on a cover slip on the same or a different mechanical arm or automatically programming to deposit a cover slip on top of the glass slide with stained sediment; m) moving using the same mechanical arm or a different mechanical arm or conveyor belt or automatically programming to move the slide with stained sediment under the microscope; n) visualizing the stained slides post staining using a standard bright field microscope with 10× and 40× magnification or automatically programming to visualize the stained slides; o) analyzing and preparing a report using a computer that has vision capabilities to visualize all the images seen by the microscope, employing several algorithms, stored data and cytopathological index are used to report the results; p) analyzing the cells after visualization of results using an algorithm “A” to identify with computer vision all the urothelial cells and other cells; q) analyzing the cells after visualization of results using an algorithm “B” for adequacy of the sample; r) analyzing the cells after visualization of results using an algorithm “C” that automatically takes photos of visual fields with abnormal cells; s) analyzing the cells after visualization of results using an algorithm “D” that count cells in all visual fields examined; t) analyzing the cells after visualization of results using an algorithm “E” that using data processing methods enters the findings of the present examination into Form “A” and stores the data; u) analyzing the cells after visualization of results using an algorithm “F” that uses data processing methods to enter the results of current examination and previous examinations into Form “B” to allow comparison of results and stores the data; v) analyzing the cells after visualization of results using an algorithm “G” makes decisions: no abnormal cells, it sends the populated Form “A” and a narrative to a printer to issue written report and to send it to the referring client, and if abnormal cells are present, it sends the photos with the abnormal cells to a pathologist for final analysis, to send commands to the mechanical arm, centrifuge, microscope, to the printers (to print labels for urine samples, tubes with urine to be centrifuged, slides, to write and print reports, to send data to a pathologist or referring client; w) analyzing the cells after visualization of results using an algorithm “H” writes reports; and x) printing using a printer the reports prepared by the method providing a result of negative for cancer risk susceptibility or to convey a probability of cancer risk susceptibility and need for further examination for which the slides and report are forwarded to an expert pathologist.

As used and disclosed herein, Form A as shown in the table 1 below provides details regarding the cells identified in the method of the present invention.

TABLE 1
FORM “A”: List of all cells that can be found
and the cells found in the current analysis.

| | | |
|---|---|---|
| | | Disposition |
| | | (a) |
| | | If all cells normal: |
| | | automatically |
| | | prints a report and |
| | | send (P + S) |
| | | (b) |
| | | If any cell |
| | | abnormal, |
| | | automatically |
| | Cells | sends the slide to |
| Cells identified | present | Pathology (STP) |

| | | |
|---|---|---|
| | UROTHELIAL CELLS | |
| 1 | Negative for urothelial cell abnormality | +S |
| | or unsatisfactory | |
| 2 | Negative for high-grade urothelial | S |
| | carcinoma | |
| 3 | Atypical urothelial cells | STP |
| 4 | Suspicious for high grade urothelial | STP |
| | carcinoma | |
| 5 | High-grade urothelial carcinoma | STP |
| 6 | Low-grade urothelial carcinoma | STP |
| 7 | Other malignancies | STP |
| 8 | NUCLEATED NON-IDENTIFIED CELLS | STP |
| | OTHER IDENTIFIED NUCLEATED CELLS | |
| 9 | Lymphocytes | P |
| 10 | Monocytes | P |
| 11 | Neutrophils | P |
| 12 | Eosinophils | P |
| 13 | Renal tubules epithelial cells | P |
| 14 | Crystals and debris | |

As used and disclosed herein, Form B as shown in the table 2 below provides details regarding comparison of the cells identified in the method of the present invention.

TABLE 2
FORM “B”: Comparison of recent and previous results.

| | Date | Date | Date | Date | Date | Date | Date | Date |
|---|---|---|---|---|---|---|---|---|
| Cells identified | (1) | (2) | (3) | (4) | (5) | (6) | (7) | (8) |

| | |
|---|---|
| | UROTHELIAL |
| | CELLS |
| 1 | Negative for urothelial |
| | cell abnormality or |
| | unsatisfactory |
| 2 | Negative for high-grade |
| | urothelial carcinoma |
| 3 | Atypical urothelial cells |
| 4 | Suspicious for high |
| | grade urothelial |
| | carcinoma |
| 5 | High-grade urothelial |
| | carcinoma |
| 6 | Low-grade urothelial |
| | carcinoma |
| 7 | Other malignancies |
| 8 | NUCLEATED NON- |
| | IDENTIFIED CELLS |
| | OTHER IDENTIFIED |
| | NUCLEATED CELLS |
| 9 | Lymphocytes |
| 10 | Monocytes |
| 11 | Neutrophils |
| 12 | Eosinophils |
| 13 | Renal tubules epithelial |
| | cells |
| 14 | Crystals and debris |

Clinical benefits of the apparatus, system, and method as disclosed in the present invention in comparison with existing methods include that: (a) the apparatus, system, and method as disclosed in the present invention fulfills a clinical need, that is, it allows to rule out the presence of urothelial carcinoma in a large number of persons, with minimal cost and medical personnel involvement. (b) The apparatus, system, and method as disclosed in the present invention shows a significant improvement over the existing methods because: (i) it automatically identifies malignant cells without the intervention of a pathologist with the use of an AI and (ii) it is a screening test and not a histopathological test, like a tissue biopsy, because it identifies cells, like in a UA but the images of abnormal cells are transmitted to the pathologist for accurate and final diagnosis. (c) If used alongside a routine UA, many asymptomatic malignancies are diagnosed among the millions of UA performed every day for other purposes. (d) It standardizes the performance of the examination and reproducibility of the results by using the same technique to prepare the samples and the same examining methods. This allows comparing current results with previous results and monitoring the evolution of disease processes and responses to treatments. (e) Images and reports are transmitted electronically to primary physicians. (f) It has a lower cost and shorter turnaround time than existing automatic methods to identify cancer cells. (g) It fulfills a clinical need and be a major innovation in the field in the last 30 years. (h) Staining the slides with Papanicolaou method has the following benefits: hematoxylin-eosin stain is still essential for cancer diagnosis and is the stain of choice to identify other cells in the sediment (44-45). Urine cytology has low sensitivity to diagnose renal cell carcinoma, and a higher sensitivity (38%) and specificity (98%) to diagnose high growth urothelial or transitional cells carcinoma but lower sensitivity to diagnose low grade tumors (3,23,5-Oeyen). However, the likelihood of these diagnoses improves in patients in whom the test is performed along a UA because the latter is periodically performed on many patients over many years for other medical reasons and if the sensitivity of the test could be improved by centrifuging a larger sample of urine, i.e. 50 mL-100 mL. In addition, if used along a routine UA it improves the diagnostic capabilities of both tests, i.e., abundant transitional epithelial cells are rarely seen in normal urine sediment and its presence requires to rule/out neoplasia or urinary tract infections (39), and if red blood cells (rbcs) or hemoglobin are identified in the sediment of a routine urinalysis, the stained sample often helps to identify the anatomical origin of hematuria; glomerulo-tubular (visualization of dysmorphic rbcs) or urinary tract (isomorphic rbcs) (46-48).

In an embodiment of the present invention, it provides a method of automatically preparing and analyzing urine samples obtained from a subject for screening the subject for cancer risk susceptibility, the method comprising the steps of: (A) providing at least one source sample 38, at least one manipulator arm 20, at least one centrifuge 21, at least one electronic microscope 22, and at least one unitary controller 23, wherein the unitary controller 23 is communicably coupled to the manipulator arm 20, the centrifuge 21, and the electronic microscope 22, wherein a cytopathological index is stored on the unitary controller 23; (B) preparing the source sample 38 into a plurality of sample tubes 45 with the manipulator arm 20, wherein each sample tube 45 includes a sample identification 39; (C) loading the sample tubes 45 into the centrifuge 21 with the manipulator arm 20; (D) executing a separation process on the sample tubes 45 with the centrifuge 21; (E) removing the sample tubes 45 from the centrifuge 21 with the manipulator arm 20; (F) extracting a plurality of sediment samples 46 with the manipulator arm 20, wherein each sample tube 45 is associated to a corresponding sediment sample 47 from the plurality of sediment samples 46; (G) preparing a plurality of sample slides 48 with the manipulator arm 20, wherein each sediment sample 46 is associated to a corresponding sample slide 48 from the plurality of sample slides 48; (H) collecting general image data 30 of each sample slide 48 with the electronic microscope 22; (I) designating a plurality of cellular contacts 31 from the general image data 30 of each sample slide 48 with the unitary controller 23; (J) assessing a cytopathological classification for each cellular contact 31 of each sample slide 48 in accordance to the cytopathological index with the unitary controller 23; and (K) generating a sample report with the unitary controller 23 by compiling the cytopathological classification for each cellular contact 31 of each sample slide 48.

In another embodiment of the present invention providing the method of automatically preparing and analyzing urine samples for identifying cancer cells as disclosed herein, wherein the step B comprises: providing at least one label generator 24, wherein the unitary controller 23 is communicably coupled to the label generator 24, wherein the manipulator arm 20 includes at least one pipette 25; retrieving a source identification for the source sample 38 with the unitary controller 23 during step B; filling each sample tube 45 with a specified volume of the source sample 38 with the pipette 25; sealing each sample tube 45 with the manipulator arm 20; compiling the source identification and the specified volume into the sample identification 39 for each sample tube 45 with the unitary controller 23; and applying a physical label 40 for the sample identification 39 of each sample tube 45 with the label generator 24.

In another embodiment of the present invention providing the method of automatically preparing and analyzing urine samples for identifying cancer cells as disclosed herein, wherein the step C goes into the step D involving: relaying a loading confirmation from the manipulator arm 20 to the unitary controller 23 after step C; generating a set of centrifugation instructions with the unitary controller 23; relaying the set of centrifugation instructions from the unitary controller 23 to the centrifuge 21; and executing the separation process in accordance to the set of centrifugation instructions with the centrifuge 21 during step D.

In another embodiment of the present invention providing the method of automatically preparing and analyzing urine samples for identifying cancer cells as disclosed herein, wherein the step E to step G progression involves: providing the manipulator arm 20 with at least one pipette 25; disposing a supernatant 50 from each sample tube 45 with the manipulator arm 20 after step E; injecting a quantity of solvent 51 into each sample tube 45 with the pipette 25 in order to dissolve a corresponding sediment sample 47 into a quantity of solvent 51 for each sample tube 45; and applying the quantity of solvent 51 with each sediment sample 46 onto the corresponding sample slide 48 with the pipette 25 during step G.

In another embodiment of the present invention providing the method of automatically preparing and analyzing urine samples for identifying cancer cells as disclosed herein, wherein the step G comprises: providing at least one label generator 24, wherein the unitary controller 23 is communicably coupled to the label generator 24, wherein the manipulator arm 20 includes at least one pipette 25; generating a slide identification 41 for each sample slide 48 with the unitary controller 23, wherein the slide identification 41 for each sample slide 48 corresponds to and is the same as the sample identification 39 for each sample tube 45 of the corresponding sediment sample 47; applying a physical label 40 for the slide identification 41 of each sample slide 48 with the label generator 24; and applying a plurality of staining solutions 52 to each sample slide 48 with the pipette 25 during step G. In yet another embodiment of the present invention providing the method of automatically preparing and analyzing urine samples for identifying cancer cells as disclosed herein, the plurality of staining solutions 52 comprises acetic acid, water, OG-6 dye, EA-50 dye, methanol, and xylene in variable concentrations and order.

In another embodiment of the present invention providing the method of automatically preparing and analyzing urine samples for identifying cancer cells as disclosed herein, wherein the step G comprises: providing at least one label generator 24 and a plurality of basins with chemicals for staining referred to as chemical basins 26, wherein the unitary controller 23 is communicably coupled to the label generator 24; generating a slide identification 41 for each sample slide 48 with the unitary controller 23, wherein the slide identification 41 for each sample slide 48 corresponds to and is the same as the sample identification 39 for each sample tube 45 for the corresponding sediment sample 47; applying a physical label 40 for the slide identification 41 of each sample slide 48 with the label generator 24; and applying a plurality of staining solutions 52 to each sample slide 48 by immersing each sample slide 48 into each chemical basin with the manipulator arm 20 during step G, wherein each staining solution is retained within a corresponding chemical basin 27 from the plurality of chemical basins 26.

In another embodiment of the present invention providing the method of automatically preparing and analyzing urine samples for identifying cancer cells as disclosed herein, wherein the step H comprises: (A′) placing a specific sample slide 48 into a field of view 28 of the electronic microscope 22 with the manipulator arm 20 during step H, wherein the specific sample slide is one selected from the plurality of sample slides 48; (B′) capturing the general image data 30 for the specific sample slide 48 with the electronic microscope 22; (C′) removing the specific sample slide 48 from the field of view 28 of the electronic microscope 22 with the manipulator arm 20; and executing a plurality of iterations for steps (A′) through (C′) for the entire plurality of sample slides 48, until all sediment samples 46 are catalogued with general image data 30 corresponding to each specific sample slide 48, wherein each sample slide 48 is designated as the specific slide in a corresponding iteration from the plurality of iterations for steps (A′) through (C′).

In another embodiment of the present invention providing the method of automatically preparing and analyzing urine samples for identifying cancer cells as disclosed herein, wherein the step I comprises: providing at least one cellular identification metric managed by the unitary controller 23; comparing the general image data 30 of each sample slide 48 to the cellular identification metric with the unitary controller 23 in order to identify at least one matching datum from the general image data 30 of each sample slide 48; and designating the matching datum as the plurality of cellular contacts 31 from the general image data 30 of each sample slide 48 with the unitary controller 23 during step I.

In another embodiment of the present invention providing the method of automatically preparing and analyzing urine samples for identifying cancer cells as disclosed herein, wherein the step J comprises: providing the cytopathological index with a plurality of classification types; comparing each cellular contact 31 from the general image data 30 of each sample slide 48 to each classification type with the unitary controller 23 in order to identify a matching type for each cellular contact 31 from the general image data 30 of each sample slide 48, wherein the matching type is from the plurality of classification types; and designating the matching type as the cytopathological classification for each cellular contact 31 from the general image data 30 of each sample slide 48 with the unitary controller 23 during step J. In yet another embodiment of the present invention providing the method of automatically preparing and analyzing urine samples for identifying cancer cells as disclosed herein, wherein the plurality of classification types comprises a group of identification comprising healthy, benign, malignant, abnormal, irregular, and unknown. In yet another embodiment of the present invention providing the method of automatically preparing and analyzing urine samples for identifying cancer cells as disclosed herein, wherein the plurality of classification types comprises a group of identification comprising: negative for epithelial cell abnormality or healthy or negative for cancer risk susceptibility, negative for cells, intermediate urothelial cells, atypical urothelial cells, high grade urothelial carcinoma, low grade papillary urothelial neoplasms, papillary urothelial neoplasm of uncertain malignant potential, low grade papillary urothelial carcinoma, squamous cell carcinoma, adenocarcinoma, small cell carcinoma, secondary neoplasms, renal cell carcinoma, prostatic carcinoma, and colonic carcinoma.

In another embodiment of the present invention providing the method of automatically preparing and analyzing urine samples for identifying cancer cells as disclosed herein, wherein the step J to step K progression involves: providing at least one external contact information stored on the unitary controller 23; collecting focused image data 37 of at least one arbitrary cellular contact with the electronic microscope 22 after step J, if the cytopathological classification of the arbitrary cellular contact is either malignant or unknown, wherein the arbitrary cellular contact is any contact from the plurality of cellular contacts 31 of each sample slide 48; appending the focused image data 37 of the arbitrary cellular contact into the sample report with the unitary controller 23 during step K; and relaying the sample report from the unitary controller 23 to the external contact information.

In another embodiment of the present invention providing the method of automatically preparing and analyzing urine samples for identifying cancer cells as disclosed herein, wherein the method further comprises: executing a plurality of iterations for steps B through K, wherein the sample report from each iteration for steps B through K is stored on the unitary controller 23; timestamping the sample report from each iteration for steps B through K with the unitary controller 23; chronologically organizing the sample report from each iteration for steps B through K in accordance to the cytopathological index into a comprehensive report with the unitary controller 23; and outputting the comprehensive report with the unitary controller 23 by sending it to a printer for printing.

In an embodiment of the present invention, it provides a method of automatically preparing and analyzing urine samples obtained from a subject for identifying cancer cells, the method comprises the steps of: (A) providing at least one source sample 38, at least one manipulator arm 20, at least one centrifuge 21, at least one electronic microscope 22, and at least one unitary controller 23, wherein the unitary controller 23 is communicably coupled to the manipulator arm 20, the centrifuge 21, and the electronic microscope 22, wherein a cytopathological index is stored on the unitary controller 23; (B) preparing the source sample 38 into a plurality of sample tubes 45 with the manipulator arm 20, wherein each sample tube 45 includes a sample identification 39; (C) loading the sample tubes 45 into the centrifuge 21 with the manipulator arm 20; (D) executing a separation process on the sample tubes 45 with the centrifuge 21; (E) removing the sample tubes 45 from the centrifuge 21 with the manipulator arm 20; (F) extracting a plurality of sediment samples 46 with the manipulator arm 20, wherein each sample tube 45 is associated to a corresponding sediment sample 47 from the plurality of sediment samples 46; (G) preparing a plurality of sample slides 48 with the manipulator arm 20, wherein each sediment sample 46 is associated to a corresponding sample slide 48 from the plurality of sample slides 48; (H) collecting general image data 30 of each sample slide 48 with the electronic microscope 22; (I) designating a plurality of cellular contacts 31 from the general image data 30 of each sample slide 48 with the unitary controller 23; (J) assessing a cytopathological classification for each cellular contact 31 of each sample slide 48 in accordance to the cytopathological index with the unitary controller 23; and (K) generating a sample report with the unitary controller 23 by compiling the cytopathological classification for each cellular contact 31 of each sample slide 48.

In another embodiment of the present invention providing the method of automatically preparing and analyzing urine samples for identifying cancer cells as disclosed herein, wherein the step B comprises: providing at least one label generator 24, wherein the unitary controller 23 is communicably coupled to the label generator 24, wherein the manipulator arm 20 includes at least one pipette 25; retrieving a source identification for the source sample 38 with the unitary controller 23 during step B; filling each sample tube 45 with a specified volume of the source sample 38 with the pipette 25; sealing each sample tube 45 with the manipulator arm 20; compiling the source identification and the specified volume into the sample identification 39 for each sample tube 45 with the unitary controller 23; and applying a physical label 40 for the sample identification 39 of each sample tube 45 with the label generator 24.

In another embodiment of the present invention providing the method of automatically preparing and analyzing urine samples for identifying cancer cells as disclosed herein, wherein the step C goes into the step D involving: relaying a loading confirmation from the manipulator arm 20 to the unitary controller 23 after step C; generating a set of centrifugation instructions with the unitary controller 23; relaying the set of centrifugation instructions from the unitary controller 23 to the centrifuge 21; and executing the separation process in accordance to the set of centrifugation instructions with the centrifuge 21 during step D.

In another embodiment of the present invention providing the method of automatically preparing and analyzing urine samples for identifying cancer cells as disclosed herein, wherein the step E to step G progression involves: providing the manipulator arm 20 with at least one pipette 25; disposing a supernatant 50 from each sample tube 45 with the manipulator arm 20 after step E; injecting a quantity of solvent 51 into each sample tube 45 with the pipette 25 in order to dissolve a corresponding sediment sample 47 into a quantity of solvent 51 for each sample tube 45; and applying the quantity of solvent 51 with each sediment sample 46 onto the corresponding sample slide 48 with the pipette 25 during step G.

In another embodiment of the present invention providing the method of automatically preparing and analyzing urine samples for identifying cancer cells as disclosed herein, wherein the step G comprises: providing at least one label generator 24, wherein the unitary controller 23 is communicably coupled to the label generator 24, wherein the manipulator arm 20 includes at least one pipette 25; generating a slide identification 41 for each sample slide 48 with the unitary controller 23, wherein the slide identification 41 for each sample slide 48 corresponds to and is the same as the sample identification 39 for each sample tube 45 of the corresponding sediment sample 47; applying a physical label 40 for the slide identification 41 of each sample slide 48 with the label generator 24; and applying a plurality of staining solutions 52 to each sample slide 48 with the pipette 25 during step G. In yet another embodiment of the present invention providing the method of automatically preparing and analyzing urine samples for identifying cancer cells as disclosed herein, the plurality of staining solutions 52 comprises acetic acid, water, OG-6 dye, EA-50 dye, methanol, and xylene in variable concentrations and order.

In another embodiment of the present invention providing the method of automatically preparing and analyzing urine samples for identifying cancer cells as disclosed herein, wherein the step G comprises: providing at least one label generator 24 and a plurality of basins with chemicals for staining referred to as chemical basins 26, wherein the unitary controller 23 is communicably coupled to the label generator 24; generating a slide identification 41 for each sample slide 48 with the unitary controller 23, wherein the slide identification 41 for each sample slide 48 corresponds to and is the same as the sample identification 39 for each sample tube 45 for the corresponding sediment sample 47; applying a physical label 40 for the slide identification 41 of each sample slide 48 with the label generator 24; and applying a plurality of staining solutions 52 to each sample slide 48 by immersing each sample slide 48 into each chemical basin with the manipulator arm 20 during step G, wherein each staining solution is retained within a corresponding chemical basin 27 from the plurality of chemical basins 26.

In another embodiment of the present invention providing the method of automatically preparing and analyzing urine samples for identifying cancer cells as disclosed herein, wherein the step H comprises: (A′) placing a specific sample slide 48 into a field of view 28 of the electronic microscope 22 with the manipulator arm 20 during step H, wherein the specific sample slide is one selected from the plurality of sample slides 48; (B′) capturing the general image data 30 for the specific sample slide 48 with the electronic microscope 22; (C′) removing the specific sample slide 48 from the field of view 28 of the electronic microscope 22 with the manipulator arm 20; and executing a plurality of iterations for steps (A′) through (C′) for the entire plurality of sample slides 48, until all sediment samples 46 are catalogued with general image data 30 corresponding to each specific sample slide 48, wherein each sample slide 48 is designated as the specific slide in a corresponding iteration from the plurality of iterations for steps (A′) through (C′).

In another embodiment of the present invention providing the method of automatically preparing and analyzing urine samples for identifying cancer cells as disclosed herein, wherein the step I comprises: providing at least one cellular identification metric managed by the unitary controller 23; comparing the general image data 30 of each sample slide 48 to the cellular identification metric with the unitary controller 23 in order to identify at least one matching datum from the general image data 30 of each sample slide 48; and designating the matching datum as the plurality of cellular contacts 31 from the general image data 30 of each sample slide 48 with the unitary controller 23 during step I.

In another embodiment of the present invention providing the method of automatically preparing and analyzing urine samples for identifying cancer cells as disclosed herein, wherein the step J comprises: providing the cytopathological index with a plurality of classification types; comparing each cellular contact 31 from the general image data 30 of each sample slide 48 to each classification type with the unitary controller 23 in order to identify a matching type for each cellular contact 31 from the general image data 30 of each sample slide 48, wherein the matching type is from the plurality of classification types; and designating the matching type as the cytopathological classification for each cellular contact 31 from the general image data 30 of each sample slide 48 with the unitary controller 23 during step J. In yet another embodiment of the present invention providing the method of automatically preparing and analyzing urine samples for identifying cancer cells as disclosed herein, wherein the plurality of classification types comprises a group of identification comprising healthy, benign, malignant, abnormal, irregular classification. In yet another embodiment of the present invention providing the method of automatically preparing and analyzing urine samples for identifying cancer cells as disclosed herein, wherein the plurality of classification types comprises a group of identification comprising: negative for epithelial cell abnormality or healthy or negative for cancer risk susceptibility, negative for cells, intermediate urothelial cells, atypical urothelial cells, high grade urothelial carcinoma, low grade papillary urothelial neoplasms, papillary urothelial neoplasm of uncertain malignant potential, low grade papillary urothelial carcinoma, squamous cell carcinoma, adenocarcinoma, small cell carcinoma, secondary neoplasms, renal cell carcinoma, prostatic carcinoma, and colonic carcinoma.

In another embodiment of the present invention providing the method of automatically preparing and analyzing urine samples for identifying cancer cells as disclosed herein, wherein the step J to step K progression involves: providing at least one external contact information stored on the unitary controller 23; collecting focused image data 37 of at least one arbitrary cellular contact with the electronic microscope 22 after step J, if the cytopathological classification of the arbitrary cellular contact is either malignant or unknown, wherein the arbitrary cellular contact is any contact from the plurality of cellular contacts 31 of each sample slide 48; appending the focused image data 37 of the arbitrary cellular contact into the sample report with the unitary controller 23 during step K; and relaying the sample report from the unitary controller 23 to the external contact information.

In another embodiment of the present invention providing the method of automatically preparing and analyzing urine samples for identifying cancer cells as disclosed herein, the method further comprises: executing a plurality of iterations for steps B through K, wherein the sample report from each iteration for steps B through K is stored on the unitary controller 23; timestamping the sample report from each iteration for steps B through K with the unitary controller 23; chronologically organizing the sample report from each iteration for steps B through K in accordance to the cytopathological index into a comprehensive report with the unitary controller 23; and outputting the comprehensive report with the unitary controller 23 by sending it to a printer for printing.

The invention will be further explained by the following Examples, which are intended to purely exemplary of the invention, and should not be considered as limiting the invention in any way.

### EXAMPLES

### Example 1

### An Exemplary Apparatus and System for Use in a Method of Screening for Cancer Risk Suspicion in a Subject Undergoing Urinalysis or Urine Analysis

This example provides an exemplary embodiment of apparatus, devices, system for use in a method of screening for cancer risk suspicion in a subject undergoing urinalysis or urine analysis involving processing and analyzing urine samples from the subject for identifying abnormal and irregular cells based on the Cytopathology Index as disclosed herein the present invention for automatically conducting the initial screening for cancer risk susceptibility before forwarding the sample and these initial results for further examination by a pathologist for instance but only for positive result subjects, or for automatically preparing a report providing the subject with negative result for cancer risk susceptibility based on urine analysis employing AI based sample processing, slide preparation, staining and microscopic examination, image capture and analysis, and report preparation by comparing captured images with stored images of the Cytopathology Index or Cytopathological Index, used interchangeably.

The automation of the preparation and initial examination processes and resultant reduction of human intervention and manual tasks reduces variance between testing procedures as a natural consequence of human error, while simultaneously increasing the feasibility of frequent testing cycles as part of a more robust diagnostic regimen.

To accomplish this, the system of the present invention utilizes at least one manipulator arm 20, at least one centrifuge 21, at least one electronic microscope 22, and at least one unitary controller 23 (Step A). The system of the present invention is further provided with at least one source sample 38. The source sample 38 is a volume of collected urine or another analyte to be processed by the present invention. The manipulator arm 20 is a conventional programmable robotic arm in the preferred embodiment, complete with a series of end-effectors and manipulator heads as may typically be found on such installations. Dedicated installations of other such robotic arms are known to be mounted to a mobile platform or other means of displacing the manipulator arm 20, particularly in implementations involving linearly arranged processing areas.

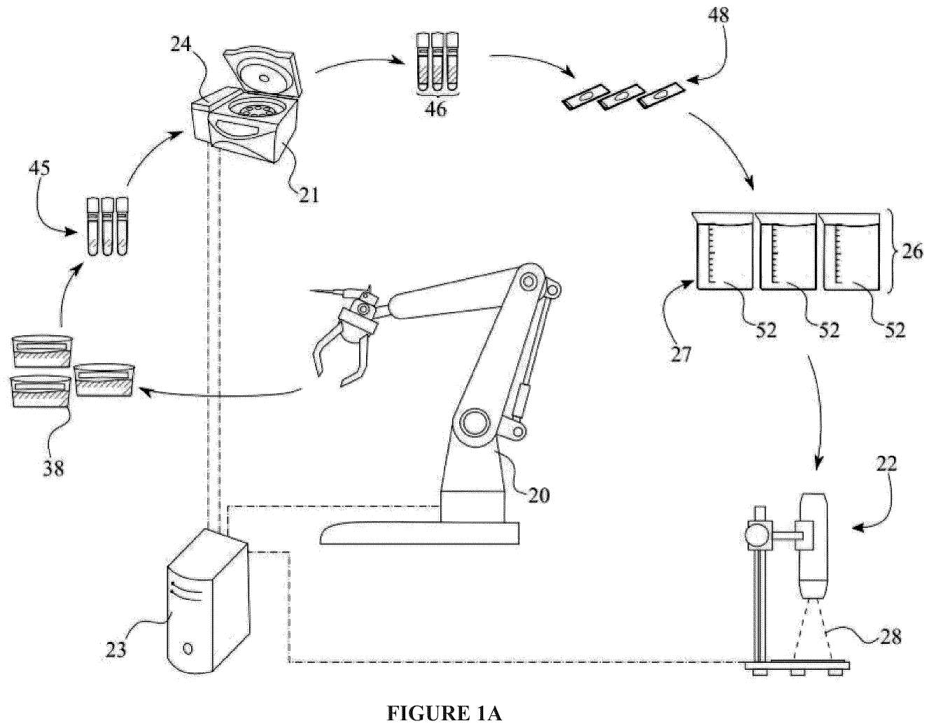

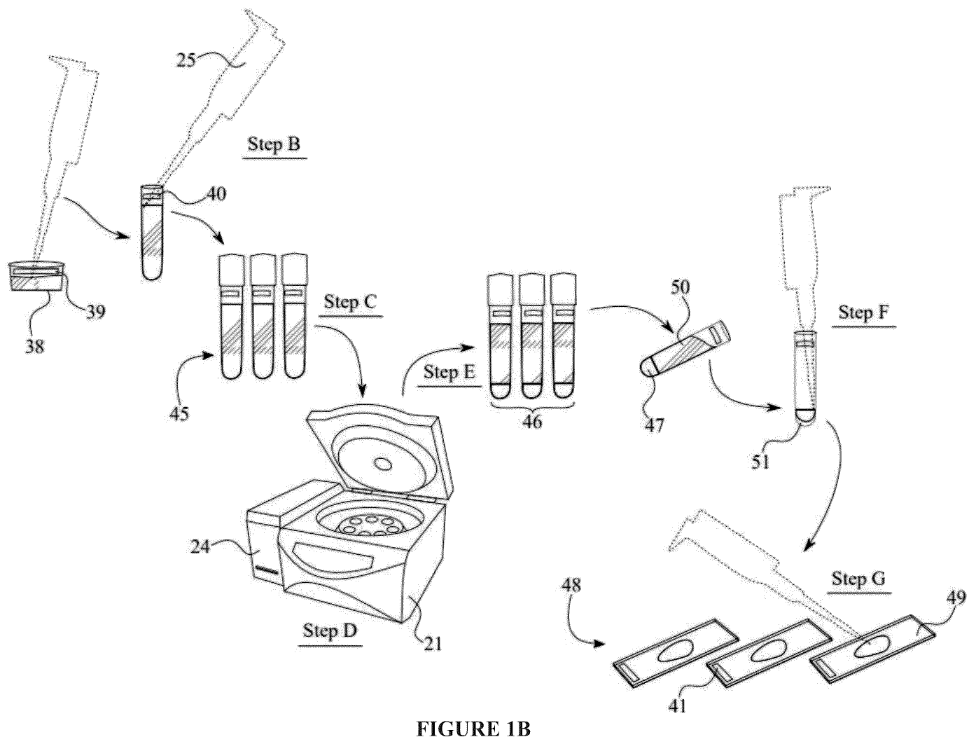

In reference to FIG. 1, the manipulator arm 20 may be centrally positioned within reach of multiple such processing areas to enable a single instance of the manipulator arm 20 to facilitate all functions of the present invention. Further, at least one manipulator arm 20 may be multiple manipulator arms 20, wherein the instances of the manipulator arm 20 are arranged to engage all other elements of the present invention cooperatively.

The centrifuge 21 constitutes a conventional separation tool 5 as may be readily available in laboratories and recognized by any suitable skilled individual. The centrifuge 21 is ideally configured for electronic control in communication with the unitary controller 23. More specifically, the operating parameters of the centrifuge 21 (rotations per minute (rpm), time in operation, start/stop commands, settle period) may be set and adjusted remotely via the unitary controller 23. Similarly, the electronic microscope 22 defines a conventional image enhancement tool that is communicably coupled to the unitary controller 23.

The electronic microscope 22 is preferably a bright light microscope with a ×10 low power field and a ×40 high power field configured to automatically capture and relay image data to the unitary controller 23 for analysis. Limitations to the type and power of the electronic microscope 22 should not be inferred from the preferred magnification power; any type of suitable magnifier or microscope may be supplemented without departing from the original spirit and scope of the present invention. Accordingly, the unitary controller 23 defines a centralized command and control system communicably coupled to the manipulator arm 20, the centrifuge 21, and the electronic microscope 22. The unitary controller 23 further defines a data processing hub suitable for image analysis and item recognition utilizing comparative analytical processes based on a cytopathological index contained therein. Additional functionalities related to out-processing of user-readable data are also supported within the unitary controller 23.

The Cytopathological Index or Cytopathology Index is a collection of interrelated reporting standards, classification thresholds, and exemplary image data that may be used to recognize, classify, and coherently describe abnormal cells captured by the microscope 22. ‘The Paris System for Reporting Urinary Cytology’ is one such element of the cytopathological index, providing a comprehensive set of terminology and diagnostic standards that may be used to effectively classify urothelial cells. Additional data may include, but is not limited to, bulk image data containing confirmed categories of atypical cells, actuarial tables relating to individual patient risk profiles, relevant medical history, or other data than may be used to inform and refine any diagnostic process performed by the present invention.

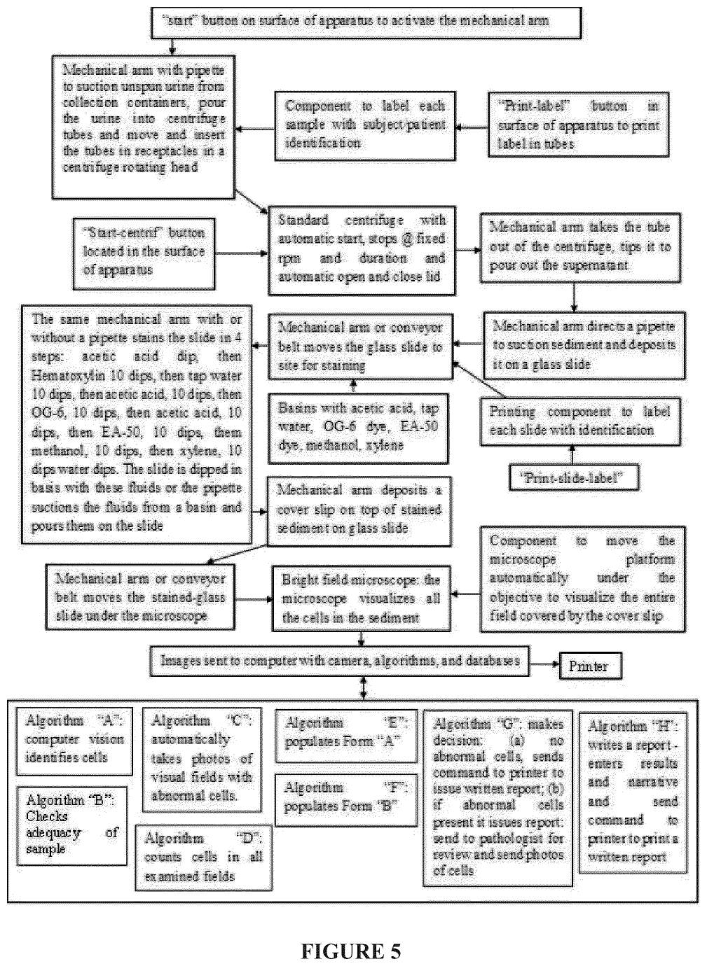

The overall process followed by the method of the present invention 5 allows the aforementioned components of the system to automatically prepare, analyze, detect, and classify atypical cells by automating the chemical staining process and employing image recognition software. Referring to FIG. 1, and FIG. 3, the overall process begins by preparing the source sample 38 into a plurality of sample tubes 45 with the manipulator arm 20, wherein each sample tube 45 includes a sample identification 39 (Step B). The plurality of sample tubes 45 defines conventional centrifugation tubes compatible with the centrifuge 21, as previously outlined. The sample identification 39 constitutes a form of readable or scannable indicia fixed to each individual sample tube 45 to ensure that the plurality of sample tubes 45 associated with an arbitrary source sample 38 are not misplaced during the overall process. As outlined in FIG. 5, each sample identification 39 will define a printed physical label 40 automatically applied by the manipulator arm 20 utilizing adhesive. In another instance, the sample identification 39 may define a programmable identifier tag associated with each sample tube 45 and encoded within the unitary controller 23. Accordingly, the unitary controller 23 will catalogue all instances within the plurality of sample tubes 45 based on their association to an original source sample 38. Preparation of the source sample 38 constitutes the targeted selection and extraction of preconfigured volumes of analyte material into testable batches to enable a single source sample 38 to provide multiple rounds of test data. Consistency of testing standards between batches is enabled by digitizing the selection and extraction process utilizing the manipulator arm 20, thereby eliminating human error inherent to a conventional manual preparation process. Consequently, resultant data sets may be analyzed with a minimized margin for error and a reduced incidence of misdiagnosis stemming from repeatability errors.

The overall process continues by loading the sample tubes 45 into the centrifuge 21 with the manipulator arm 20 (Step C), wherein the sample tubes 45 are individually seated within corresponding receptacles of the centrifuge 21 to ensure proper operation of the centrifuge 21. In reference to FIG. 6, once loading is complete the unitary controller 23 signals the centrifuge 21 to close and seal the operating hatch over the plurality of sample tubes 45 prior to beginning the centrifugation cycle. At least one implementation of the present invention may utilize a centrifuge 21 without an integral automatic closure mechanism. In this instance, the manipulator arm 20 may be configured to close the operating hatch.

Once secured, the overall process continues with executing a separation process on the sample tubes 45 with the centrifuge 21 (Step D). This separation process is defined as a centrifugation process, wherein the particles of the solution contained within the sample tubes 45 are separated according to size, shape, density, viscosity, and programmable rotor speed of the centrifuge 21. The operating parameters of the centrifuge 21 are stored within the unitary controller 23 as machine-readable instructions communicated to the centrifuge 21. Such operating parameters may include, but are not limited to, rotor speed, process duration, resting cycles, or any other metrics that may guide the execution of the separation process.

After the separation process is complete, the overall process continues by removing the sample tubes 45 from the centrifuge 21 with the manipulator arm 20 (Step E). This step is conducted as a reversal of the loading process, either individually removing sample tubes 45 or extracting an entire batch of a plurality of sample tubes 45 simultaneously before proceeding.

Subsequently, the overall process continues by extracting a plurality of sediment samples 46 with the manipulator arm 20, wherein each sample tube 45 is associated to a corresponding sediment sample 47 from the plurality of sediment samples 46 (Step F). The plurality of sediment samples 46 defines the collected testable particulate matter separated from the source sample 38 material during centrifugation. In the preferred implementation of the present invention, the plurality of sediment samples 46 will contain urothelial cells divisible into multiple diagnostic categories based on visually identifiable features. According to the internal documentation methods outlined thus far, the unitary controller 23 digitally associates the sediment sample 46 to the sample tube 45, then to the source sample 38 in a hierarchal format.

According to this hierarchal structure, the overall process continues by preparing a plurality of sample slides 48 with the manipulator arm 20, wherein each sediment sample 46 is associated to a corresponding sample slide 49 from the plurality of sample slides 48 (Step G). The plurality of sample slides 48 refers to a series of conventional transparent specimen carriers configured to mount within the field of view 28 of the electronic microscope 22. The association between sediment sample 46 and sample slide 48 may be denoted on each sample slide 48 with an additional printable tag or indicator to ensure that multiple batches of source samples 38 being processed through the system are not misidentified or cross-contaminated in later stages of operation.

The next stage of the overall process begins with collecting general image data 30 of each sample slide 48 with the electronic microscope 22 (Step H). This general image data 30 defines a relatively low-magnification view of the target sample slide 48 suitable for cursory analysis and processing to determine areas of interest for more intensive imaging and analysis in later steps.

Accordingly, the overall process continues by designating a plurality of cellular contacts 31 from the general image data 30 of each sample slide 48 with the unitary controller 23 (Step I). The plurality of cellular contacts 31 defines a machine-generated list of possible zones within the general image data 30 that are identified as containing urothelial cells and therefore may require more investigation to determine malignancy. This analysis is performed by the unitary controller 23, wherein the unitary controller 23 serves as a graphics processing unit.

The intensive imaging and investigation processes are subsequently associated with assessing a cytopathological classification for each cellular contact of each sample slide 48 in accordance to the cytopathological index with the unitary controller 23 (Step J). The cytopathological index defines a uniform machine-readable series of thresholds for identifying and reporting urothelial cells based on visually observable characteristics, i.e. size, shape, opacity, geometric complexity, or other standards as may be known to a reasonably skilled individual. The cytopathological classification for each cellular contact is thus a uniform reporting code for a profile defined by the cytopathological index, ideally categorizing each cellular contact as ‘healthy’, ‘benign’, ‘malignant’, or possibly ‘unknown’ if no suitable classification may be attached. These categories are exemplary of a preferred implementation; however, imitations to the type and descriptors of the classifications should not be implied.

The overall process concludes by generating a sample report with the unitary controller 23 by compiling the cytopathological classification for each cellular contact of each sample slide 48 (Step K). The sample report ideally contains a tabulation of all identifiable cellular contacts 31, including adjacent copies of sample reports generated for previous batches of source sample 38. The rendering of this data may include graphical representations of the volume of each cytopathological classification detected within multiple sequential batches to aid in a diagnosis of disease progression over time. The sample report may additionally include a preliminary machine-generated diagnosis based on the assessed presence and levels of various classifications within a given source sample 38.

One subprocess for the method of the present invention is used to properly label and prepare the plurality of sample tubes 45 to prevent misattribution of testing results across multiple concurrent batches or patients. Thus, the subprocess is provided with at least one label generator 24, wherein the unitary controller 23 is communicably coupled to the label generator 24, and wherein the manipulator arm 20 includes at least one pipette 25. The label generator 24 may define any form of printer, laser engraver, or configurable ink stamp that may visually mark lab materials. The unitary controller 23 provides operating instructions to the label generator 24, as with the centrifuge 21 and manipulator arm 20, to ensure effective cooperation between the disparate hardware comprising the system of the present invention. In addition, the manipulator arm 20 includes at least one pipette 25 in this instance. The pipette 25 constitutes a means of extracting a programmable volume of liquid, including any solute or suspended particulate material in said volume. To enable rapid processing of large quantities of testable material and maintain tool sterility, at least one pipette 25 may be positioned in a battery arrangement and/or equipped with self-sterilization functions as may be readily apparent to an individual skilled in the art. This subprocess begins by retrieving a source identification from the source sample 38 with the unitary controller 23 during Step B. The source identification defines both a printed indicator and a digital counterpart to the printed indicator that are unique to the source sample 38. In one instance, the manipulator arm 20 may facilitate the acquisition of the source identification from the source sample 38 with an integrated visual scanner or equivalent article. This subprocess continues by filling each sample tube 45 with a specified volume of the source sample 38 with the pipette 25. The specified volume refers to a testable amount of analyte as defined within the unitary controller 23. As previously outlined, the pipette 25 is directed to extract a consistent amount of analyte per operation to ensure that testing protocols and standards are maintained across multiple iterations of testing. This subprocess continues by sealing each sample tube 45 with the manipulator arm 20. The manipulator arm 20 is typically considered to utilize conventional caps or stoppers for this function, but any form of impermeable seal may be applied here. The unitary controller 23 subsequently compiles the source identification and the specified volume into the sample identification 39 for each sample tube 45, wherein the data corresponding to each of these distinct entries is attached to a retrievable record within the unitary controller 23. The collection of all testing data in this manner may enable the review and analysis of procedures to ensure adherence to test protocols in conjunction with a larger quality assurance program and established practices within the field. In accordance with this objective, the label generator 24 applies a physical label 40 for the sample identification 39 of each sample tube 45. The physical label 40 enables a sample to be manually tracked through the method of the present invention as a secondary measure in conjunction with the integrated tracking elements of the unitary controller 23. In practice, this feature enables the periodical spot-checking of the present invention by comparing the information written to the physical label 40 to the corresponding entries in the unitary controller 23.

Another subprocess enables the digitization of the operation of the centrifuge 21. This subprocess begins by relaying a loading confirmation from the manipulator arm 20 to the unitary controller 23 after Step C. The loading confirmation constitutes a digital signal produced at the centrifuge 21 upon closure of an access panel or lid, verifying that a complete load of the plurality of sample tubes 45 is properly mounted within the centrifuge 21. Subsequently, the appropriate separation operation must be executed according to presets stored within the unitary controller 23, which is accomplished by generating a set of centrifugation instructions with the unitary controller 23 then relaying the set of centrifugation instructions from the unitary controller 23 to the centrifuge 21. The set of centrifugation instructions may constitute a single data package of executable instructions readable in series by the centrifuge 21 or may be delivered in sequence by the unitary controller 23 dependent on the instruction buffer of the centrifuge 21. The overall process continues by executing the separation process in accordance to the set of centrifugation instructions with the centrifuge 21 during Step D. The unitary controller 23 will ideally moderate the separation process to ensure consistent and effective operation of the centrifuge 21 per testing standards. As outlined previously, the unitary controller 23 may remotely adjust rotations per minute, time in operation, start/stop commands, settle periods, or any other aspect of centrifugation as may be realized by a reasonably skilled individual.

Another subprocess allows waste material to be removed post-centrifugation prior to rendering the sediment samples 46 for inspection. This subprocess provides the manipulator arm 20 with at least one pipette 25, wherein the pipette 25 disposes of a supernatant 50 from each sample tube 45 with the manipulator arm 20 after Step E. The supernatant 50 conventionally describes any post-separation liquid residue in the plurality of sample tubes 45. This supernatant 50 is distinct from the sediment samples 46, wherein the sediment samples 46 are retained for processing and inspection as outlined previously. Next, this subprocess continues by injecting a quantity of solvent 51 into each sample tube 45 with the pipette 25 in order to dissolve the corresponding sediment sample 47 into the quantity of solvent 51 for each sample tube 45. More specifically, a known quantity of solvent 51 is applied to each of the plurality of sample tubes 45 to ensure that the entirety of each sediment sample 46 is removed from the corresponding sample tube 45. Rendering the sediment sample 46 and the solvent 51 as a composite solution enables the lossless transfer of the sediment sample 46 to a subsequent media. Accordingly, this subprocess concludes by applying the quantity of solvent 51 with each sediment sample 46 onto the corresponding sample slide 49 with the pipette 25 during Step G.

Another subprocess allows the sediment samples 46 to be chemically stained with a pipette 25. This subprocess is provided with at least one label generator 24, wherein the unitary controller 23 is communicably coupled to the label generator 24, and wherein the manipulator arm 20 includes at least one pipette 25. The subprocess continues by applying a physical label 40 for the slide identification 41 of each sample slide 48 with the label generator 24, similar to the subprocess by which a similar label may be applied to the individual sample tubes 45, previously. By extending the labelling process across all media, the integrity of any testable batches may be assured. In the event of misplacement, disordering, or loss of any testable media, the unitary controller 23 may register the loss and alert operators as appropriate. In this subprocess, chemical stains are applied directly by the manipulator arm 20 via the pipette 25. Specifically, this subprocess continues by applying a plurality of staining solutions 52 to each sample slide 48 with the pipette 25 during Step G. The plurality of staining solutions 52 is specifically contemplated to comprise acetic acid, water, OG-6 dye, EA-50 dye, methanol, and xylene in variable concentrations and order. The conventional Papanicolaou (“Pap”) stain process is considered useful for exemplary purposes, however, the specific composition and application of the plurality of staining solutions 52 is suggested to be variable across multiple embodiments without departing from the original spirit and scope of the present invention.

As an alternative to the previous subprocess, another subprocess allows the sediment samples 46 to be chemically stained with multiple bathing basins 26. This subprocess is provided with at least one label generator 24 and a plurality of chemical basins 26, wherein the unitary controller 23 is communicably coupled to the label generator 24. Similar to the previous subprocess, this subprocess begins by generating a slide identification 41 for each sample slide 48 with the unitary controller 23, wherein the slide identification 41 for each sample slide 48 is the sample identification 39 for each sample tube 45 for the corresponding sediment sample 47. Again, similar to the previous subprocess, this subprocess continues by applying a physical label 40 for the slide identification 41 of each sample slide 48 with the label generator 24. Consistent labelling throughout the overall process is considered essential to prevent misattribution of test results between patients. A simple error at this stage may have far-reaching effects on ultimate patient outcomes if the data used to inform treatment decisions is presented erroneously. The manipulator arm 20 then applies a plurality of staining solutions 52 to each sample slide 48 by immersing each sample slide 48 into each chemical basin with the manipulator arm 20 during Step G, wherein each staining solution is retained within a corresponding chemical basin 27 from the plurality of chemical basins 26. The plurality of chemical basins 26 ideally defines a series of segmented liquid containers of appropriate dimensions to accept the plurality of sample slides 48, internally. This arrangement also includes any internal supporting structures or retainers that may capture the individual sample slide 48 during the staining process. In an ideal arrangement, the plurality of chemical basins 26 is aligned according to the preferred application order of the staining process. In this arrangement, the corresponding chemical basin 27 is always immediately adjacent to the sample slide 48 slated for immersion in said corresponding chemical basin 27.

Another subprocess allows for automatic visual analysis the is supported by dedicated hardware suitable for automation (e.g. the electronic microscope 22). This subprocess begins by placing a specific sample slide 48 into a field of view 28 of the electronic microscope 22 with the manipulator arm 20 during Step H, wherein the specific sample slide 48 is from the plurality of sample slides 48 (Step A′). The field of view 28 is physically defined as the targeted focal point of the electronic microscope 22 and is digitally defined by the unitary controller 23 as a transformation of the position of the manipulator arm 20. More specifically, the field of view 28 may define an origin from which movement orders are defined, enabling repeated returns to this origin point as both an essential function of the overall system and an error mitigation method. After the specific sample slide 48 is positioned within the field of view 28, this subprocess continues by capturing the general image data 30 for the specific sample slide 48 with the electronic microscope 22 (Step B′). The general image data 30 constitutes a broad overview of the specific sample slide 48 and the corresponding sediment sample 47 contained therein for the purposes of establishing an initial diagnosis. The initial diagnosis generated by the unitary controller 23 catalogues items of interest within the general image data 30 for later analysis, ideally performed based on a triage of the items of interest performed at this stage. This subprocess continues by removing the specific sample slide 48 from the field of view 28 of the electronic microscope 22 with the manipulator arm 20 (Step C′) to clear the field of view 28 for a subsequent sample slide 48. Accordingly, this subprocess is repeated by executing a plurality of iterations for Step A′ through C′, wherein each sample slide 48 is designated as the specific slide in a corresponding iteration from the plurality of iterations for Step A′ through Step C′. This reiteration is continued for the entire plurality of sample slides 48, until all sediment samples 46 are catalogued with general image data 30 corresponding to each specific sample slide 48.

Another subprocess allows for the automatic visual identification of medically significant cellular conditions. This subprocess is provided with at least one cellular identification metric that is managed by the unitary controller 23. The cellular identification metric defines an established standard for differentiating between various archetypes of a cell based on visual characteristics. In various conceivable applications, any singular cellular identification metric may be applied in conjunction with multiple distinct cellular identification metrics to generate a robust diagnosis based on established confidence thresholds for said visual characteristics. This subprocess begins by comparing the general image data 30 of each sample slide 48 to the cellular identification metric with the unitary controller 23 in order to identify at least one matching datum from the general image data 30 of each sample slide 48. The matching datum defines any characteristic determined to be within the thresholds for positive inclusion to any class or category of cellular contact. Accordingly, this subprocess continues by designating the matching datum as the plurality of cellular contacts 31 from the general image data 30 of each sample slide 48 with the unitary controller 23 during Step I. In practice, the matching datum is compiled with other instances of matching datum and utilized to compile a list of inclusive categories, whereby said categories may be weighted during analysis to determine final placement of the corresponding cellular contact into a simplified classification. This simplified classification, or preliminary diagnosis, is attached to each individual cellular contact within the unitary controller 23. Consequently, each cellular contact may be reviewed within an accessible database as opposed to manually reacquiring said cellular contact within an archived sample slide 48 or article of general image data 30.

After general image data 30 is captured for each specific sample slide 48, another subprocess allows for in-depth analysis to extract actionable data from the broader data sets outlined previously. The actionable data is defined in relation to a reporting standard for cellular contacts 31, specifically urothelial cells and visual characteristics thereof.

Conventional laboratory testing requires a technician to visually identify these urothelial cells post-stain to determine the type and quantity of cellular contacts 31 present within a sample. Provisions are made to enable the unitary controller 23 to automatically apply these standards in order to provide the Cytopathological Index with a plurality of classification types. These classification types may be attached to more simplistic monikers for ease of use, such as ‘healthy’, ‘benign’, ‘malignant’, or other labels as previously outlined including unknown. However, the plurality of classification types is understood to encompass all identifiable characteristics of cells as may be visually ascertained, including a detailed description of these characteristics behind any simplified labels to be stored within the unitary controller 23. This subprocess begins by comparing each cellular contact from the general image data 30 of each sample slide 48 to each classification type with the unitary controller 23 in order to identify a matching type for each cellular contact from the general image data 30 of each sample slide 48, wherein the matching type is from the plurality of classification types. Automatic identification and classification of each cellular contact enables the present invention to perform diagnostic processes from initial sample preparation through data interpretation, minimizing the manpower requirements for routine testing. Further, the digitization of the plurality of classification types enables the collection of training data that may be used to refine the automatic identification processes in an iterative learning environment. This subprocess finishes by designating the matching type as the cytopathological classification for each cellular contact from the general image data 30 of each sample slide 48 with the unitary controller 23 during step J. This refinement will enable a diagnostician or attending physician to rapidly assess the cellular contacts 31 present within the general image data 30, consequently accelerating the testing procedure and removing barriers to additional testing that may otherwise render such efforts prohibitive.

Another subprocess allows the present invention to disseminate data to medical professionals in an effort to collaboratively diagnose a patient using said data. In this embodiment, the present invention may integrate opinions and recommendations from external sources to modify and improve the automatic image recognition system at the core of the present invention. This subprocess is provided with at least one external contact information stored on the unitary controller 23. The external contact information may be an email address, phone number, or internal identifier for any telemedicine software application compatible with the unitary controller 23. The external contact information is preferably prerecorded within the unitary controller 23 as ancillary data attached to the source sample 38, identifying a supervising physician or person otherwise responsible for applying the test results in a broader context. As shown in exemplary form in FIG. 3, this subprocess continues by collecting focused image data 37 of at least one arbitrary cellular contact with the electronic microscope 22 after Step J, if the cytopathological classification of the arbitrary cellular contact is either malignant or unknown, wherein the arbitrary cellular contact is any contact from the plurality of cellular contacts 31 of each sample slide 48. The focused image data 37 defines localized image data centered around cellular contacts 31 determined to fall within categorizations other than ‘healthy’ during previous analyses of the general image data 30. This subprocess generally presumes that cellular contacts 31 recognized as non-problematic with a high degree of confidence need not be targeted for additional investigation, though the focused image data 37 may be captured by direction or policy as configured by a user. Separating each cellular contact by location within the general image data 30 enables the electronic microscope 22 to target the arbitrary cellular contact with higher magnification, enabling the unitary controller 23 to capture and analyze the arbitrary cellular contact for characteristics that may have been obscured or otherwise unclear in the general image data 30. Once the focused image data 37 relating to the arbitrary cellular contact is captured and analyzed, this subprocess concludes by appending the focused image data 37 of the arbitrary cellular contact into the sample report with the unitary controller during Step K and then relaying the sample report from the unitary controller 23 to the external contact information. The pre-filtering of image data serves to reduce time spent on non-critical analyses, freeing additional time for in-demand medical professionals to perform in-depth analyses as required. Further, the automatic and direct presentation of said image data reduces organizational inefficiencies stemming from lost or non-standard communiques between medical and administrative staff.

The present invention is proposed to be beneficial as both a standalone process and a tool for generating consistent time-scaled diagnostics and assessments of extended courses of treatment. As shown in FIG. 4, a plurality of iterations for Step B through Step K are executed so that the sample report from each iteration for Step B through Step K is stored on the unitary controller 23. The sample report is retained by the unitary controller 23 for continuous comparison to future iterations of the overall process, whereby insights into disease progression and treatment efficacy may be gained from a record. Supporting this file arrangement, the unitary controller 23 timestamps the sample report from each iteration for Step B through Step K and then chronologically organizes the sample report from each iteration for Step B through Step K in accordance to the Cytopathological Index into a comprehensive report. Appending an indelible time reference to the sample report enables all data contained within the sample report to be automatically modelled over time by conventional data handling software. In the preferred embodiment, the comprehensive report includes an evolving data set stored within the unitary controller 23. This comprehensive report supports the inclusion of all historical medical data (physicians notes, observational reports, admission records, toxicology screens, etc.) as contemporaneous appendages to the data generated in relation to the plurality of cellular contacts 31. Utilizing this data, a physician can expect to map trends in patient condition to forecast future developments. Though the comprehensive report is preferably contained by the unitary controller 23, it is suggested that multiple instances of the comprehensive report corresponding to a single patient may be maintained remotely via persistent updates from the unitary controller 23. Finally, the unitary controller 23 outputs the comprehensive report, wherein an output of the comprehensive report may be digital and/or physical copies of the conclusions and supporting data.

### Example 2

### Another Exemplary Apparatus and System for Use in a Method of Screening for Cancer Risk Suspicion in a Subject Undergoing Urinalysis or Urine Analysis

This example provides another exemplary embodiment of apparatus, devices, system for use in a method of screening for cancer risk suspicion in a subject undergoing urinalysis or urine analysis involving processing and analyzing urine samples from the subject for identifying abnormal and irregular cells based on the Cytopathology Index as disclosed herein the present invention for automatically conducting the initial screening for cancer risk susceptibility before forwarding the sample and these initial results for further examination by a pathologist for instance but only for positive result subjects, or for automatically preparing a report providing the subject with negative result for cancer risk susceptibility based on urine analysis employing AI based sample processing, slide preparation, staining and microscopic examination, image capture and analysis, and report preparation by comparing captured images with stored images of the Cytopathology Index or Cytopathological Index, used interchangeably as provided in FIG. 5.

As shown in the flowchart of FIG. 5, the method as disclosed commences by the pushing of a “start” button on surface of apparatus to activate the mechanical arm, followed by use of a mechanical arm with pipette to suction unspun urine from collection containers, pour the urine into centrifuge tubes and move and insert the tubes in receptacles in a centrifuge rotating head, while a “Print-label” button is pushed or programmed via a unitary controller i.e., a computer, in surface of apparatus as disclosed herein to print label in tubes, where a component to label each sample with subject/patient identification. Next, either manual pushing or automated program via the unitary controller or the computer of a “Start-centrif” button located in the surface of apparatus leads to automatic operation of a standard centrifuge with automatic start, which stops after centrifuging at a fixed rotation per minutes (rpm) and after a fixed duration and with incorporation of automatic open and close lid functions. Then, either the same or another mechanical arm or a conveyor belt takes the tube out of the centrifuge, tips it to pour out the supernatant and then either the same or another mechanical arm directs a pipette to suction sediment and deposits it on a glass slide. Next, either the same or another mechanical arm or a conveyor belt moves the glass slide to site for staining, where said mechanical arm with or without a pipette stains the slide in 4 steps: first an acetic acid dip, then in Hematoxylin-10 dips, then in tap water -10 dips, then again in acetic acid-10 dips, then in OG-6 dye-10 dips, then again in acetic acid-10 dips, then in EA-50 dye-10 dips, then in methanol-10 dips, then in xylene-10 dips, and lastly again in water dips. The slide is dipped in basins with these fluids or the pipette suctions the fluids from a basin and pours them on the slide, and then the mechanical arm deposits a cover slip on top of stained sediment on glass slide. Next, either the same or another mechanical arm or a conveyor belt moves the stained-glass slide under the microscope, where the microscope is a bright field microscope that visualizes all the cells in the sediment, where a component to move the microscope platform automatically under the objective to visualize the entire field covered by the cover slip. Finally, the captured images that are visualized and captured under the microscope with a camera are assessed and analyzed by various algorithms and databases and compared with stored data using artificial intelligence (AI) utilizing comparative analytical processes based on a cytopathological index contained in the computer or unitary controller. The algorithms in this step include: Algorithm “A” for identifying and classifying cells based on computer vision; Algorithm “B” for checking adequacy of the sample processed in the method as disclosed herein; Algorithm “C” for automatically taking photos of visual fields with abnormal cells; Algorithm “D” that counts cells in all examined fields; Algorithm “E” that populates Form “A”; Algorithm “F” that populates Form “B”; Algorithm “G” that makes decision that can be either (a) no abnormal cells, it sends command to printer to issue written report; or (b) if abnormal cells present, it issues a report that there is a cancer risk susceptibility and hence a need to send the sample and report for further examination to a pathologist for review and according to send photos of cells captured in the method as disclosed herein; and finally Algorithm “H” that writes a report-enters results and narrative and send command to printer to print a written report.

It will be apparent to those skilled in the art that various modifications and variations can be made in the practice of the present invention without departing from the scope or spirit of the invention. Other embodiments of the invention will be apparent to those skilled in the art from considering of the specification and practice of the invention. It is intended that the specification and examples be considered as exemplary only, with a true scope and spirit of the invention being indicated by the following claims.

### REFERENCES

1. https://www.cancer.org/research/cancer-facts-statistics/all-cancer-facts-figures/cancer-facts-figures-2018.html
2. Antoni S, Fertay J, Soerjomataram I, Znaor A, Jemal A, Bray F, Bladder cancer incidence and mortality: A global overview and recent trends. Eur Urol 2017 January; 71 (1): 96.
3. Palaoro LA, Angerose M, Correlation between the cytology of urine sediment in fresh sample and smears stained by Papanicolaou and Giemsa methods, J Cytol 2014 January-Mar; 31 (1): 25-31.
4. Planz B, Jochima E, Deix T, Caspers H P, Jakse G, Boecking A. The role of urinary cytology for detection of bladder cancer, Eur J Surg Oncol. 2005 April; 31 (3): 304-8.
5. Davis N, Mor Y, Idelevich P, Terkieltaub D, Siv V, Elkeles A, Lew S, Okon E, Laufer M, Ramon J, Kedar D, Baniel J, Yossepovitch O, A novel urine cytology stain for the detection and monitoring of bladder cancer, J Urol 2014 December; 192 (6): 1628-32.
6. Xing J, Reynolds, JP, Diagnostic Advances in Urine Cytology. Surg Pathol Clin 2018 September; 11 (3): 601-610.
7. Oeyen E, Hoekx L, De Wachter S, Baldewijns M, Ameye F, Mertens I, Bladder Cancer Diagnosis and Follow-Up: The Current Status and Possible Role of Extracellular Vesicles. Int J Mol Sci. 2019 Feb. 14; 20 (4).
8. Gupta M, VandenBussche C J, Bivalacqua T J, Urinary cytology and the Paris system for reporting urinary pathology: implications for urological management, Cytopathology 2018 August; 29 (4): 368-370.
9. Roy M, Kaushal S, Jain D, Seth A, Iyer V K, Mathur SR, An institutional experience with The Paris system: A paradigm shift from ambiguous terminology to more objective criteria for reporting urine cytology. Cytopathology. 2017 December; 28 (6): 509-515.
10. Barkan GA, Wojcik EM, Nayar R, Savic-Ptrince S, Quek ML, Kurtycz D F, Rosenthal DL, The Paris System for Reporting Urinary Cytology: The quest to develop a standardized terminology. Acta Cytol. 2016; 60 (3): 185-197.
11. Zheng X, Si Q, Du D, Harshan M, Zhang Z, Haines K, Shi W, Chieng DC, The Paris System for urine cytology in upper tract urothelial specimens: A comparative analysis with biopsy and surgical resection. Cytopathology. 2018 April; 29 (2): 184-188.
12. Rohilla M, Sing P, Rajwanshi A, Gupta N, Srivinasan R, Day P, Kakker N, Cytohistological correlation of urine cytology in a tertiary center with application of the Paris System. Cytopathology. 2018 October; 29 (5): 436-443.
13. Kurtycz DFI, Barkan GA, Pavelec DM, Rosenthal DL, Wojcik EM, VandenBussche CJ, Mangiulli K, Olson MT, Paris Interobserver Reproducibility Study (PIRST), J Am Soc Cytopathol 2018 July-Aug; 7 (4) 174-180.
14. Miki Y, Neat M, Chandra A, Application of the Paris System to atypical urine cytology samples: correlation with histology and UroVysion FISH. Cytopathology. 2017 April; 28 (2): 88-95.
15. Tetu B, Diagnosis of urothelial carcinoma from urine, Mod Pathol. 2009 Ju; 22 Suppl 2: S53-9.
16. Mowatt G, Zhu S, Kilonzo M, Boachie C, Fraser C, Griffiths TR, N′Dow J, Nabi G, Cook J, Vale L, Systematic review of the clinical effectiveness and cost-effectiveness of photodynamic diagnosis and urine biomarkers (GISH, ImmunoCyt, NMP22) and cytology for the detection and follow-up of bladder cancer, Health Technol Assess. 2010 January; 14 (4): 1-331.
17. Lavery H J, Zaharieva B, McFaddin A, Heerema N, Pohar K S, A prospective comparison of UroVysion FISH and urine cytology in bladder cancer detection. BMC Cancer. 2017; 17:247.
18. Yang T, Li Y, Li J, Liu J, Deng X, Wang G, Diagnostic Value Comparison of Urothelium Carcinoma Among Urine Exfoliated Cells Fluorescent in Situ Hybridization (FISH) Examination, Computerized Tomography (CT) Scan, and Urine Cytologic Examination, Med Sci Mont. 2018 Aug. 19; 24:5788-5792.
19. Schmitz-Drager C, Bonberg N, Pesch B, Todenhofer T, Sahin S, Behrens T, Bruning T, Schmitz-Drager BJ. Replacing cystoscopy by urine markers in the follow up of patients with low risk non-muscle invasive bladder cancer?—An International Bladder Cancer Network Project, Urol Oncol. 2016 October; 34 (10): 452-9.
20. Odisho AY Berry AB, Ahmad AE, Cooperberg MR, Carroll PR, Konety BR, Reflex immunocytology testing for the diagnosis of bladder cancer in patients with atypical urine cytology, Eur Urol. 2013 May; 63 (5): 936-40.
21. Martnes-Uzunova E S, Bottcher R, Croce C M, Jenster G, Visakorpi T, Calin GA, Long Noncoding RNA in prostate, bladder, and Kidney, Eur Urol. 2014 June; 65 (6): 1140-51.
22. Lin SY, Linehan JA, Wilson GT, Hoon DSB, Emerging Utility of Urinary Cell-free Nuclei Acid Biomarkers for Prostate, Bladder, and Renal Cancers, Eur Urol Focus. 2017 April; 3 (2-3): 265-272).
23. Wiener HG, Remkes GW, Schatzl G, Susani M, Breitenecker G, Quick-staining urinary cytology and bladder wash image analysis with an integrated risk classification: a worthwhile improvement in the follow-up of bladder cancer? Cancer. 1999 Oct. 25; 87 (5): 263-9.
24. Barkan GA Enough is enough: Adequacy of voided urine cytology, Cancer Cytopathology, 2016; 124 (3) 163-166.
25. VandenBussche CJ, Rosenthal DL, Olson MT, Adequacy in voided urine cytology specimens: The role of volume and a repeat void upon predictive values for high-grade urothelial carcinoma, Cancer Cytopathology, 2016; 124 (3): 174-180.
26. Fisher AH, Jacobson KA, Rose J, Zeller R, Hematoxylin and Eosin staining of tissue and cell sections, CSH Protoc. 2008 May 1: pdb.prot4986.
27. Schumann GB, Weiss MA, Atlas of Renal and Lower Urinary Track cytology and histopathologic basis. Philadelphia, Lippincott, 1981, pp 1-287.
28. Kiel DP, Moskowitz MA, The Urinalysis: A Critical Appraisal, Medical Clinics of North America, Vol 71, No 4, July 1987.
29. Ashman JJ, Rui P, Okeyode T, Characteristics of Office-based Physician Visits, 2015, CDC NCHS, Ambulatory care use and physician office visits NCHS date brief No 310, June 2018.
30. Summerville JA, Maxted W C, Pahira J J, Urinalysis: A comprehensive Review, Am Family Phys, Volume 71, No 6, Mar. 15, 2005, 1153-1162.
31. Corey Cavanaugh, Mark A Perazella, Urine sediment examination in the diagnosis and management of kidney disease: Core Curriculum 2019.
32. Dumler F, Rocher L L, Madrazo B, Diagnostic Evaluations of the Kidney and the Urinary Tract, in Clinical Nephrology, Dialysis and Transplantation, 2009, Edit. Malluche HH, Sawaya BP, Hakim RM, Sayegh MH, pp 1-11.
33. Papanicolaou (PAP) Staining: Introduction, Principle, Procedure and Interpretation, https://laboratoyr info.com, July 2018.
34. Sternheimer R, A Supravital Cytodiagnostic Stain for Urinary Sediments, JAMA 1975; 231 (8): 826-832.
35. Zogno C, Schiaffino E, Boeri R, Schmidt C, Cytologic detection of metastatic malignant melanoma in urine. A report of three cases. Acta Cytol 1997 July-Aug; 41 (4 Suppl): 1332-6.
36. Kyung S K, Ji Young Yang, Jae Ho Han, Ji Eun Kwon, Metastatic Blue Nevus-Like Melanoma Detect by Liquid-Based Catheterized Urine Cytology, Ann Dermatol. 2018 June; 30 (3): 356-360.
37. Diaz-Peromingo J A, Tato-Rodriguez J, Pesqueira-Fontan P M, Molonos-Castro S, Gayol-Fernandez MC, Struzik JP, non-Hodgkin's lymphoma presenting as a primary bladder tumor: a case report. J Med Case Reports, 2010; 4:114.
38. Kodzo-Grey Venyo A, Lymphoma of the Urinary Bladder, Ad Urol. 2014; 2014:327917.
39. Hamid R T, Pantanowitz L, Artificial intelligence and digital pathology: Challenges and opportunities, J Pathol Inform 2018, 9:38.
40. Pantanowitz L, Parwani A V. Digital pathology. ASCP Press; 2017. p 304. ISBN: 978-08189-6104.
41. Van Laak J, Rajpoot N, Voseen D. The promise of computation pathology: Part 1. The pathologist. 2018a. https://thepathologist.com.
42. Van Laak J, Rajpoot N, Vossen D. The promise of computational pathology: Part 2. The pathologist. 2018b. https://thepatholgoist.com.
43. Litjens G, Kooi T, Bejnordi B E, Setio A A, Ciompi F, Ghafoorian M. A survey on deep learning in medical image analysis. Med Image Anal 2017; 42:60-88.
44. Jha S, Topol E J. Adapting to artificial intelligence: Radiologist and pathologist as information specialists. JAMA 2016; 316:2353-4.
45. Kayser K, Gortler J, Bogovac M, Bogovac A, Goldmann T, Voolmer E, Kayser G. AI (artificial intelligence) in histopathology—from image analysis to automated diagnosis, Folia Histochimica et Cytobiologica Vol 47, No. 3, 2009, pp 355-361.
46. Fogazzi GB, Ponticelli C, Ritz E, The Urinary sediment: An Integrated View, Milano, Italy: Elsevier Masson; 2010.
47. Ali S Z, Rosenthal DL, Epstein JI, Ali TZ Atlas of Urinary Cytopathology, with Histopathologic correlations. Springer Publishing, 2009.
48. Rosenthal DL, Wojcik EM, Kurtycz DFI, eds. The Paris System for Reporting Urinary Cytology, New York: Springer; 2016.
49. Nikas I, Reynolds J P et al. The Paris System for Reporting Urinary Cytology: A Meta-Analysis J Pers Med. 2022 February; 12 (2): 170).

What is claimed is:

1. A method of screening for cancer risk suspicion in a subject or patient undergoing urine analysis or urinalysis referred to as UA, the method comprising the steps of:
a) employing an apparatus comprising a mechanical arm with a pipette to automatically suction a fixed volume of unspun urine sample from a collection container, and pour it into a centrifuge tube and insert the centrifuge tube with the urine sample into one of the receptacles of a centrifuge rotating head, said step optionally is performed manually;
b) activating by a first button labelled start that is located on the surface of the apparatus to activate the mechanical arm when pressed;
c) activating by a second button labelled a print-label that is located on the surface of the apparatus to automatically print a label with each subject identification on each centrifuge tube with the urine sample or optionally manually write on the tube, wherein the printing is done before the tube is inserted in a centrifuge rotating head and while the tubes are held by the mechanical arm, wherein the information to be printed is typed in by an attached keyboard of a first computer and printed on each centrifuge tube with the urine sample when pressing the print-label button or optionally manually written, and optionally, a printed or hand-written label is attached manually to the centrifuge tube with the urine sample;
d) transferring the centrifuge tube with the urine sample into a centrifuge automatically, the centrifuge comprising: (i) a rotating head with multiple receptacles to house 12-48 centrifuge tubes, wherein each receptacle accommodates tubes of different sizes, and (ii) electro-mechanical components to allow different speeds, rotation per minute (rpm) and duration of centrifugation, wherein the centrifuge is used to separate blood and urine;
e) centrifuging the centrifuge tube with the urine sample automatically, wherein the centrifuge automatically closes the lid, starts and stops at pre-set rpms for a fixed duration of centrifugation, and then automatically opens the lid after centrifugation is completed, wherein step e is started by pressing a third button labelled start-centrifuge located on the surface of the apparatus, and optionally, all the steps c to e are automatically run without having to press the print-label and start-centrifuge buttons;
f) taking out the centrifuge tube with the urine sample after centrifugation in step e is completed automatically by the mechanical arm of step a or by a different mechanical arm, tilting the tube to pour out the supernatant and tilting the tube back to upright position to keep the urine sediment inside the centrifuge tube, wherein step f starts automatically after the centrifuge stops and the lid opens in step e;
g) injecting into the centrifuge tube, a small and fixed volume of saline or other liquid automatically, by a pipette attached to the mechanical arm of step a or a different mechanical arm to re-dissolve the sediment left at the bottom of the centrifuge tube in step f to obtain a re-dissolved sediment in the centrifuge tube;
h) suctioning a fixed volume of the re-dissolved sediment in the centrifuge tube and depositing it on a glass slide automatically;
i) printing by a printing component on the glass slide of step h automatically, a label with the subject identification, wherein this information is printed by pressing a fourth button labelled print-slide-label located on the surface of the apparatus or optionally by a sent key on the keyboard of the first computer, and optionally, this step is done automatically after the step h without having to type and press the print-slide-label button;
j) moving the glass slide labelled in step i automatically by the mechanical arm of step a or by a different mechanical arm or by a conveyor belt to a site for staining the sediment deposited on the glass slide;
k) staining the glass slide with the Papanicolaou method at the site for staining automatically either by dipping the glass slide into basins with several fluids or dyes, or by a pipette suctioning these fluids or dyes from the basin and pouring them on top of the slide; 1) depositing a cover slip on top of the glass slide of step k with the stained sediment automatically by the mechanical arm of step a or by a different mechanical arm, wherein the cover slip covers the expanse of the stained sediment, and optionally, this step is done manually; m) moving the glass slide with the stained sediment covered by the cover slip from step 1 automatically by the mechanical arm of step a or by a different mechanical arm or by a conveyor belt under a microscope for visualization of all areas covered by the cover slip, wherein the mechanical arm or the conveyor belt moves the glass slide under the microscope automatically or on command to help the visualization by the microscope of all areas of all cells present and stained on the glass slide and capturing pictures of images visualized; n) analyzing the pictures captured from step m under the second computer with a software comprising various algorithms, the second computer comprising content of stored data including: a Cytopathology Index which is a library or database with thousands of images of all cells that are found in the sample; o) removing the glass slide with the mechanical arm of step a or by a different mechanical arm or by a conveyor belt under the microscope after the visualization in step m is completed and depositing it in a storage box for any further examination, and optionally, this step is done manually; p) analyzing with an adequacy algorithm “B” for assessing the proper relationship between specimen source, cytological diagnosis, urine volume, urothelial cellularity, and obscuring features, wherein the obscuring features include non-urothelial cells including vaginal contaminants, bacteria, acute inflammation, sperm, and crystal, wherein said obscuring features in higher amounts obscure the findings and characterization of urothelial cells; q) analyzing with an algorithm “C” for automatically taking photos of each visual field and storing them; r) analyzing with an algorithm “D” for automatically counting the number of each cell in all visual fields examined;
s) analyzing with an algorithm “E” that automatically uses data processing methods to enter the findings of the present examination into form “A” and stores the data; t) analyzing with an algorithm “F” that automatically uses data processing methods to enter the findings of the present and previous examinations into form “B” and stores the data; u) analyzing with an algorithm “G” that automatically makes decisions and decides how to proceed after the slide examination is completed, wherein the decisions are selected from a group consisting of: (i) when no abnormal cells are seen, send a command to print a report and send it to the referring physician (P+S) reporting negative cancer risk suspicion, and (ii) when abnormal or atypical or unidentified cells are seen, to send a command to send the slides to an expert pathologist for final review (STP) and reporting the same on probability of cancer risk suspicion; v) employing an algorithm “H” to automatically write a report based on the report in step u, wherein the said report includes patient or subject name, date of birth, sex selected from a group consisting of male and female, previous examination history as a yes or no, referral history, detail on type of specimen, wherein the type can be selected from a group consisting of voided, instrumented, and ileal conduit urine, an adequacy statement, wherein the adequacy statement provides information selected from a group consisting of satisfactory for evaluation statement, wherein the statement provides volume, collection type, cellularity, and cytomorphological findings, and unsatisfactory for evaluation statement, wherein the statement provides information providing reasons including small volume and not enough cells, or recognizable cells, hypercellularity because of acute inflammation; and w) sending the report from step v for printing automatically by a printer.

2. The method of screening for cancer risk suspicion of claim 1, wherein the staining procedure in step k comprises the steps of: (1) automatically dipping the glass slide into an acetic acid basin by the mechanical arm of step a or by a different mechanical arm, or optionally the mechanical arm of step a or by a different mechanical arm using a pipette to automatically suction acetic acid from a container and pouring it on top of the glass slide; (2) dipping the glass slide by the mechanical arm of step a or by a different mechanical arm into a water basin for rinsing, or optionally the mechanical arm of step a or by a different mechanical arm using a pipette to automatically suction water from the basin and pour it on top of the slide for rinsing; (3) dipping the glass slide by the mechanical arm of step a or by a different mechanical arm into a water basin for rinsing or optionally the mechanical arm of step a or by a different mechanical arm using a pipette to automatically suction water from the basin and pour it on top of the glass slide for rinsing, (4) dipping the glass slide by the mechanical arm of step a or by a different mechanical arm into an acetic acid basin or optionally the mechanical arm of step a or by a different mechanical arm using a pipette to automatically suction acetic acid and pour it on top of the glass slide; (5) dipping the glass slide by the mechanical arm of step a or by a different mechanical arm into a OG-6 (dye) basin or optionally the mechanical arm of step a or by a different mechanical arm using a pipette to automatically suction this dye from a basin and pour it on top of the glass slide; (6) dipping the glass slide by the mechanical arm of step a or by a different mechanical arm into a EA-50 (dye) basin or optionally the mechanical arm of step a or by a different mechanical arm using a pipette to automatically suction this dye from a basin and pour it on top of the glass slide; (7) dipping the glass slide by the mechanical arm of step a or by a different mechanical arm into a methanol basin, or optionally the mechanical arm of step a or by a different mechanical arm using a pipette to automatically suction methanol from a basin and pour it on top of the glass slide; (8) dipping the glass slide by the mechanical arm of step a or by a different mechanical arm into a xylene basin or optionally the mechanical arm of step a or by a different mechanical arm using a pipette to automatically suction xylene and pour it on top of the glass slide; (9) dipping the glass slide by the mechanical arm of step a or by a different mechanical arm into a tap water basin or by a different mechanical arm using a pipette to automatically suction tap water and pour it on top of the glass slide for rinsing, and optionally, the staining of samples is done manually, wherein the slides are manually inserted into the apparatus for cytological examination.

3. The method of screening for cancer risk suspicion of claim 1, wherein the visualization in step m is done under a bright field microscope, and wherein the bright field microscope comprising: (aa) lenses to give a magnification of ×10 (low power field), ×40 (high power field), and lenses to give a larger magnification for better visualization of nucleolus in search for cancer cells, and (bb) a platform where the glass slide is deposited that moves automatically in a pre-arranged manner to allow visualization of different positions covering all the sediment covered areas under the cover slip, wherein in each of the positions, the images of cells present are sent to a second computer for analysis with a photo taken of said positions, wherein the motion of the platform starts immediately after the slide is deposited on it, wherein the glass slide is first scanned under the ×10 magnification objective and next under the ×40 magnification objective, and optionally, the area of the slide covered with the glass cover could be visualized with a grid to be able to identify the areas where abnormalities are noted.

4. A method of screening for cancer risk suspicion in a subject or patient undergoing urinalysis or urine analysis, the method comprising the steps of:
a) suctioning automatically a fixed volume of unspun urine from a collection container a mechanical arm with a pipette of an apparatus to pour it in a centrifuge tube and move and insert the tube in a receptacle in a centrifuge rotating head;
b) activating the mechanical arm by hitting a button labelled “start”, located on the surface of the apparatus;
c) printing automatically with a printing component to label each centrifuge tube with each subject or patient identification;
d) processing automatically with a centrifuge with internal mechanics to automatically close the lid, start and stop at pre-set rotation per minutes referred to as rpms and duration of centrifugation, and open the lid after the centrifugation is completed;
e) activating the centrifuge with a button labelled “start-centrif” to start centrifugation, which can be programmed to be activated automatically by a computer;
f) moving automatically the tubes using the same mechanical arm or a different mechanical arm to automatically take the tubes out of the centrifuge, tilt them to pour out the supernatant and tilt them back to upright position keeping inside the tube the urine sediment;
g) injecting automatically using the same mechanical arm takes a pipette or a different mechanical arm with a pipette to automatically inject a small and fixed volume of saline or other liquid into the tube to re-dissolve the sediment left at the bottom of the tube, suctions a fixed volume of re-dissolved sediment from the bottom of each centrifuge tube and deposit it on a glass slide;
h) labeling using a printing component to label each slide with the patient identification printed in the centrifuge tubes;
i) pushing or automatically programming a button labelled “Print-label-slide” which is to be pushed, and it is located on the surface of the apparatus to initiate printing of the label in the glass slide;
j) moving using the same mechanical arm or a different mechanical arm or a conveyor belt or automatically programming to move the glass slide to a site where the sediment is stained;
k) staining using the same mechanical arm that takes a pipette or a different mechanical arm or automatically programming to stain the slide with the Papanicolaou method in four steps by either dipping the slide into different basins containing different fluids or dyes or holding a pipette to suction these fluids/dyes and pour them on top of the slide, wherein basins with different fluids/dyes are used for staining the slide;
l) putting on a cover slip on the same or a different mechanical arm or automatically programming to deposit a cover slip on top of the glass slide with stained sediment;
m) moving using the same mechanical arm or a different mechanical arm or conveyor belt or automatically programming to move the slide with stained sediment under the microscope;
n) visualizing the stained slides post staining using a standard bright field microscope with 10× and 40× magnification or automatically programming to visualize the stained slides;
o) analyzing and preparing a report using a computer that has vision capabilities to visualize all the images seen by the microscope, employing several algorithms, stored data and cytopathological index are used to report the results;
p) analyzing the cells after visualization of results using an algorithm “A” to identify with computer vision all the urothelial cells and other cells;
q) analyzing the cells after visualization of results using an algorithm “B” for adequacy of the sample;
r) analyzing the cells after visualization of results using an algorithm “C” that automatically takes photos of visual fields with abnormal cells;
s) analyzing the cells after visualization of results using an algorithm “D” that count cells in all visual fields examined;
t) analyzing the cells after visualization of results using an algorithm “E” that using data processing methods enters the findings of the present examination into Form “A” and stores the data;
u) analyzing the cells after visualization of results using an algorithm “F” that uses data processing methods to enter the results of current examination and previous examinations into Form “B” to allow comparison of results and stores the data;
v) analyzing the cells after visualization of results using an algorithm “G” makes decisions: no abnormal cells, it sends the populated Form “A” and a narrative to a printer to issue written report and to send it to the referring client, and if abnormal cells are present, it sends the photos with the abnormal cells to a pathologist for final analysis, to send commands to the mechanical arm, centrifuge, microscope, to the printers (to print labels for urine samples, tubes with urine to be centrifuged, slides, to write and print reports, to send data to a pathologist or referring client;
w) analyzing the cells after visualization of results using an algorithm “H” writes reports; and
x) printing using a printer the reports prepared by the method providing a result of negative for cancer risk susceptibility or to convey a probability of cancer risk susceptibility and need for further examination for which the slides and report are forwarded to an expert pathologist.

5. A method of automatically preparing and analyzing urine samples obtained from a subject for screening the subject for cancer risk susceptibility, the method comprising the steps of:
(A) providing at least one source sample 38, at least one manipulator arm 20, at least one centrifuge 21, at least one electronic microscope 22, and at least one unitary controller 23, wherein the unitary controller 23 is communicably coupled to the manipulator arm 20, the centrifuge 21, and the electronic microscope 22, wherein a cytopathological index is stored on the unitary controller 23;
(B) preparing the source sample 38 into a plurality of sample tubes 45 with the manipulator arm 20, wherein each sample tube 45 includes a sample identification 39;
(C) loading the sample tubes 45 into the centrifuge 21 with the manipulator arm 20;
(D) executing a separation process on the sample tubes 45 with the centrifuge 21;
(E) removing the sample tubes 45 from the centrifuge 21 with the manipulator arm 20;
(F) extracting a plurality of sediment samples 46 with the manipulator arm 20, wherein each sample tube 45 is associated to a corresponding sediment sample 47 from the plurality of sediment samples 46;
(G) preparing a plurality of sample slides 48 with the manipulator arm 20, wherein each sediment sample 46 is associated to a corresponding sample slide 48 from the plurality of sample slides 48;
(H) collecting general image data 30 of each sample slide 48 with the electronic microscope 22;
(I) designating a plurality of cellular contacts 31 from the general image data 30 of each sample slide 48 with the unitary controller 23;
(J) assessing a cytopathological classification for each cellular contact 31 of each sample slide 48 in accordance to the cytopathological index with the unitary controller 23; and
(K) generating a sample report with the unitary controller 23 by compiling the cytopathological classification for each cellular contact 31 of each sample slide 48.

6. The method of automatically preparing and analyzing urine samples obtained from a subject for screening the subject for cancer risk susceptibility of claim 5, wherein the step B comprises: providing at least one label generator 24, wherein the unitary controller 23 is communicably coupled to the label generator 24, wherein the manipulator arm 20 includes at least one pipette 25; retrieving a source identification for the source sample 38 with the unitary controller 23 during step B; filling each sample tube 45 with a specified volume of the source sample 38 with the pipette 25; sealing each sample tube 45 with the manipulator arm 20; compiling the source identification and the specified volume into the sample identification 39 for each sample tube 45 with the unitary controller 23; and applying a physical label 40 for the sample identification 39 of each sample tube 45 with the label generator 24.

7. The method of automatically preparing and analyzing urine samples obtained from a subject for screening the subject for cancer risk susceptibility of claim 5, wherein the step C goes into the step D involving: relaying a loading confirmation from the manipulator arm 20 to the unitary controller 23 after step C; generating a set of centrifugation instructions with the unitary controller 23; relaying the set of centrifugation instructions from the unitary controller 23 to the centrifuge 21; and executing the separation process in accordance to the set of centrifugation instructions with the centrifuge 21 during step D.

8. The method of automatically preparing and analyzing urine samples obtained from a subject for screening the subject for cancer risk susceptibility of claim 5, wherein the step E to step G progression involves: providing the manipulator arm 20 with at least one pipette 25; disposing a supernatant 50 from each sample tube 45 with the manipulator arm 20 after step E; injecting a quantity of solvent 51 into each sample tube 45 with the pipette 25 in order to dissolve a corresponding sediment sample 47 into a quantity of solvent 51 for each sample tube 45; and applying the quantity of solvent 51 with each sediment sample 46 onto the corresponding sample slide 48 with the pipette 25 during step G.

9. The method of automatically preparing and analyzing urine samples obtained from a subject for screening the subject for cancer risk susceptibility of claim 5, wherein the step G comprises: providing at least one label generator 24, wherein the unitary controller 23 is communicably coupled to the label generator 24, wherein the manipulator arm 20 includes at least one pipette 25; generating a slide identification 41 for each sample slide 48 with the unitary controller 23, wherein the slide identification 41 for each sample slide 48 corresponds to and is the same as the sample identification 39 for each sample tube 45 of the corresponding sediment sample 47; applying a physical label 40 for the slide identification 41 of each sample slide 48 with the label generator 24; and applying a plurality of staining solutions 52 to each sample slide 48 with the pipette 25 during step G. In yet another embodiment of the present invention providing the method of automatically preparing and analyzing urine samples for identifying cancer cells as disclosed herein, the plurality of staining solutions 52 comprises acetic acid, water, OG-6 dye, EA-50 dye, methanol, and xylene in variable concentrations and order.

10. The method of automatically preparing and analyzing urine samples obtained from a subject for screening the subject for cancer risk susceptibility of claim 5, wherein the step G comprises: providing at least one label generator 24 and a plurality of basins with chemicals for staining referred to as chemical basins 26, wherein the unitary controller 23 is communicably coupled to the label generator 24; generating a slide identification 41 for each sample slide 48 with the unitary controller 23, wherein the slide identification 41 for each sample slide 48 corresponds to and is the same as the sample identification 39 for each sample tube 45 for the corresponding sediment sample 47; applying a physical label 40 for the slide identification 41 of each sample slide 48 with the label generator 24; and applying a plurality of staining solutions 52 to each sample slide 48 by immersing each sample slide 48 into each chemical basin with the manipulator arm 20 during step G, wherein each staining solution is retained within a corresponding chemical basin 27 from the plurality of chemical basins 26.

11. The method of automatically preparing and analyzing urine samples obtained from a subject for screening the subject for cancer risk susceptibility of claim 5, wherein the step H comprises: (A′) placing a specific sample slide 48 into a field of view 28 of the electronic microscope 22 with the manipulator arm 20 during step H, wherein the specific sample slide is one selected from the plurality of sample slides 48; (B′) capturing the general image data 30 for the specific sample slide 48 with the electronic microscope 22; (C′) removing the specific sample slide 48 from the field of view 28 of the electronic microscope 22 with the manipulator arm 20; and executing a plurality of iterations for steps (A′) through (C′) for the entire plurality of sample slides 48, until all sediment samples 46 are catalogued with general image data 30 corresponding to each specific sample slide 48, wherein each sample slide 48 is designated as the specific slide in a corresponding iteration from the plurality of iterations for steps (A′) through (C′).

12. The method of automatically preparing and analyzing urine samples obtained from a subject for screening the subject for cancer risk susceptibility of claim 5, wherein the step I comprises: providing at least one cellular identification metric managed by the unitary controller 23; comparing the general image data 30 of each sample slide 48 to the cellular identification metric with the unitary controller 23 in order to identify at least one matching datum from the general image data 30 of each sample slide 48; and designating the matching datum as the plurality of cellular contacts 31 from the general image data 30 of each sample slide 48 with the unitary controller 23 during step I.

13. The method of automatically preparing and analyzing urine samples obtained from a subject for screening the subject for cancer risk susceptibility of claim 5, wherein the step J comprises: providing the cytopathological index with a plurality of classification types; comparing each cellular contact 31 from the general image data 30 of each sample slide 48 to each classification type with the unitary controller 23 in order to identify a matching type for each cellular contact 31 from the general image data 30 of each sample slide 48, wherein the matching type is from the plurality of classification types; and designating the matching type as the cytopathological classification for each cellular contact 31 from the general image data 30 of each sample slide 48 with the unitary controller 23 during step J.

14. The method of automatically preparing and analyzing urine samples obtained from a subject for screening the subject for cancer risk susceptibility of claim 5, wherein the step J to step K progression involves: providing at least one external contact information stored on the unitary controller 23; collecting focused image data 37 of at least one arbitrary cellular contact with the electronic microscope 22 after step J, if the cytopathological classification of the arbitrary cellular contact is either malignant or unknown, wherein the arbitrary cellular contact is any contact from the plurality of cellular contacts 31 of each sample slide 48; appending the focused image data 37 of the arbitrary cellular contact into the sample report with the unitary controller 23 during step K; and relaying the sample report from the unitary controller 23 to the external contact information.

15. The method of automatically preparing and analyzing urine samples obtained from a subject for screening the subject for cancer risk susceptibility of claim 5, wherein the method further comprises: executing a plurality of iterations for steps B through K, wherein the sample report from each iteration for steps B through K is stored on the unitary controller 23; timestamping the sample report from each iteration for steps B through K with the unitary controller 23; chronologically organizing the sample report from each iteration for steps B through K in accordance to the cytopathological index into a comprehensive report with the unitary controller 23; and outputting the comprehensive report with the unitary controller 23 by sending it to a printer for printing.
